(19) United States
(12) Patent Application Publication
(10) Pub. No.: US 2026/0020966 A1
(43) Pub. Date: Jan. 22, 2026

(54) MUSCULOSKELETAL IMPLANT WITH SENSORS

(71) Applicant: GLOBUS MEDICAL, INC., AUDUBON, PA (US)

(72) Inventors: Paden Troxell, Conshohocken, PA (US); David C. Paul, Phoenixville, PA (US); Norbert Johnson, North Andover, MA (US)

(21) Appl. No.: 18/776,377

(22) Filed: Jul. 18, 2024

(51) Int. Cl. A61F 2/46; A61F 2/28; A61F 2/30

(52) U.S. Cl. CPC A61F 2/4657; A61F 2002/2821; A61F 2002/3067; A61F 2002/4666; A61F 2002/469

(57) ABSTRACT

A musculoskeletal implant device includes at least one sensor, processing circuitry, and a memory containing instructions executable by the processing circuitry. The musculoskeletal implant device is operable to obtain sensor information indicating measurements taken by the sensor connected to the musculoskeletal implant device during or after implantation of the musculoskeletal implant device into a patient. The musculoskeletal implant device is further operable to provide feedback for controlling modification of a feature associated with the musculoskeletal implant device based on the sensor information.

### FIELD

The present disclosure relates to medical implants, including musculoskeletal implants.

### BACKGROUND

A goal of musculoskeletal surgery is to improve, manage, or treat symptoms and provide stability to the musculoskeletal system. These procedures can range from spine fusion, deformity correction, vertebral augmentation, fracture fixation, and joint arthroplasty. Implantable medical devices are often placed during surgical procedures to assist in achievement of treatment goals. Existing implant technologies, which are often purely mechanical in nature, are approaching a limit of clinical effectiveness. Innovations have come in waves of new materials like PEEK and TAV, integration with navigation and robotics systems, manufacturing process improvements like 3D printing, mechanical design optimizations, and advanced coatings. However, investments in many of these engineering enhancements are reaching diminishing returns with respect to patient outcomes.

Historically, surgeons only could rely on their 5 senses to operate in a safe and effective manner. Over the past 30 years, the introduction of imaging, navigation, robotics, and neuromonitoring technologies have provided surgeons with additional feedback mechanisms that enable more accurate, repeatable, and minimally invasive procedures.

### SUMMARY

Embodiments of the present disclosure are directed to smart musculoskeletal implants that can provide physicians/clinicians and their patients the ability to monitor, detect, and diagnose different features

Some embodiments of the present disclosure are directed to a method performed by a musculoskeletal implant device. The method includes obtaining sensor information indicating measurements taken by a sensor connected to the musculoskeletal implant device during or after implantation of the musculoskeletal implant device into a patient. The method further includes providing feedback for controlling modification of a feature associated with the musculoskeletal implant device based on the sensor information.

Some other embodiments of the present disclosure are directed to a musculoskeletal implant device that includes at least one sensor; processing circuitry; and a memory containing instructions executable by the processing circuitry. The musculoskeletal implant device is operable to obtain sensor information indicating measurements taken by the sensor connected to the musculoskeletal implant device during or after implantation of the musculoskeletal implant device into a patient. The musculoskeletal implant device is further operable to provide feedback for controlling modification of a feature associated with the musculoskeletal implant device based on the sensor information.

Some other embodiments of the present disclosure are directed to a method performed by a medical system for controlling modification of a musculoskeletal implant device. The method includes receiving, from the musculoskeletal implant device, sensor information indicating measurements taken by a sensor connected to the musculoskeletal implant device during or after implantation of the musculoskeletal implant device into a patient. The method further includes transmitting modification instructions for controlling modification of a feature associated with the musculoskeletal implant device based on the sensor information.

### DESCRIPTION OF THE DRAWINGS

Aspects of the present disclosure are illustrated by way of example and are not limited by the accompanying drawings. In the drawings:

FIG. 1 illustrates an example of a smart musculoskeletal implant that includes a traditional musculoskeletal implant and a control and communication module, according to some embodiments of the present disclosure;

FIG. 2 illustrates an example of a control and communication module that includes a control subsystem, communication subsystem, and power subsystem, according to some embodiments of the present disclosure;

FIGS. 3-4 illustrate a control subsystem, according to some embodiments of the present disclosure

FIG. 5 illustrates a smart musculoskeletal implant implanted in a patient's body and is communicatively coupled with a non-implanted external device and another smart musculoskeletal implant implanted in the patient's body, according to some embodiments of the present disclosure;

FIG. 6 illustrates a smart musculoskeletal implant implanted in a patient's body and includes a physically separate but communicatively connected component that is also implanted in a patient's body which is used to communicate with a non-implanted external device, according to some embodiments of the present disclosure;

FIG. 7 illustrates a power subsystem within the smart musculoskeletal implant that is implanted in a patient's body, according to some embodiments of the present disclosure;

FIG. 8 illustrates a power subsystem of a smart musculoskeletal implant that includes an energy harvesting device and rechargeable battery that capture energy from an ambient environment, according to some embodiments of the present disclosure;

FIG. 9 illustrates another power subsystem of a smart musculoskeletal implant that includes an energy harvesting device and rechargeable battery that capture energy from an energy receiver component that is physically separate from the smart musculoskeletal implant, according to some embodiments of the present disclosure;

FIG. 10 illustrates a power subsystem of the smart musculoskeletal implant that is configured to receive transmitted energy from an external device, according to some embodiments of the present disclosure;

FIG. 11 illustrates another power subsystem of the smart musculoskeletal implant that is configured to receive transmitted energy from an energy receiver component that is physically separate from the smart musculoskeletal implant, according to some embodiments of the present disclosure;

FIG. 12 illustrates periods throughout the continuum of care in which embodiments can be used;

FIG. 13 illustrates a connected care system, according to some embodiments of the present disclosure;

FIG. 14 illustrates an operating room that includes a smart implant and smart instruments that are communicatively connected to each other, according to some embodiments of the present disclosure;

FIG. 15 illustrates a smart implant and smart instrument that is configured to provide real-time (or near real-time) feedback, according to some embodiments of the present disclosure;

FIG. 16 illustrates another operating room that includes a smart implant and an enabling technology, according to some embodiments of the present disclosure;

FIG. 17 illustrates another smart implant and another smart instrument that is configured to provide real-time (or near real-time) feedback to enabling technology, according to some embodiments of the present disclosure;

FIG. 18 illustrates an embodiment of intraoperative connectivity that includes a smart implant connected to an enabling technology, which is connected to a cloud, according to some embodiments of the present disclosure;

FIG. 19 illustrates an embodiment of intraoperative connectivity that includes a smart implant connected to an enabling technology via a smart implant controller device, according to some embodiments of the present disclosure

FIG. 20 illustrates yet another embodiment of intraoperative connectivity that includes two smart implants connected to an enabling technology via a smart implant controller device, according to some embodiments of the present disclosure;

FIG. 21 illustrates a smart implant configured to communicate sensor information to an enabling technology via a controller (e.g., clinician controller), according to some embodiments of the present disclosure;

FIG. 22 illustrates yet another embodiment of intraoperative connectivity that includes a primary smart implant and a secondary smart implant connected to an enabling technology via a smart implant controller device, according to some embodiments of the present disclosure;

FIG. 23 illustrates a hospital/clinic environment where a smart implant provides feedback to a clinician, according to some embodiments of the present disclosure;

FIG. 24 illustrates a patient's home environment where a smart implant provides sensor information to a clinician via a patient smart implant controller and/or a patient smart phone, according to some embodiments of the present disclosure;

FIG. 25 illustrates a smart implant providing data/power to another smart implant that provides the smart implant's and the another smart implant's data/power to a wearable for providing feedback to a clinician, according to embodiments of the present disclosure;

FIG. 26 illustrates a smart musculoskeletal implant that includes a screw capable of sensing the type of tissue that is in contract with, or in proximity to, the screw, according to some embodiments of the present disclosure;

FIG. 27 illustrates a musculoskeletal implant device that includes 24 integrated force sensors enable real-time mapping of loading on the endplates, according to some embodiments of the present disclosure;

FIG. 28 illustrates independent expansion of anterior and posterior elements (IAP) of the musculoskeletal implant device with real-time feedback on implant loading, according to some embodiments of the present disclosure;

FIG. 29 illustrates a visualization of feedback regarding pressure exerted on a musculoskeletal implant device in real-time, according to some embodiments of the present disclosure;

FIG. 30 illustrates another visualization of feedback regarding pressure exerted on a musculoskeletal implant device in real-time, according to some embodiments of the present disclosure;

FIG. 31 illustrates load paths through a musculoskeletal implant device that includes strain gauge sensors within or on the musculoskeletal implant device, according to some embodiments of the present disclosure;

FIG. 32 illustrates a top sectional view and a side view of an interbody spacer with electrodes spaced periodically along a surface of a graft window in a single plane, according to some embodiment of the present disclosure;

FIG. 33 illustrates a top sectional view of an interbody spacer that sequentially uses different electrodes to measure the filling of graft material, according to some embodiments of the present disclosure;

FIG. 34 illustrates a top sectional view of another interbody spacer that sequentially uses different electrodes to measure the filling of graft material, according to some embodiments of the present disclosure;

FIG. 35 illustrates a top sectional view of yet another interbody spacer that uses different electrodes to measure the filling of graft material and determine the location of a gap in the graft material, according to some embodiments of the present disclosure;

FIG. 36 illustrates an example of a two-dimensional map of tissue within a graft window, according to embodiments of the present disclosure;

FIG. 37 illustrates a side section view of a multiplanar electrode array, according to some embodiments of the present disclosure;

FIG. 38 illustrates an example of a two-dimensional map and a three-dimensional map of tissue within a graft window, according to some embodiments of the present disclosure;

FIG. 39 illustrates an application of a smart implant for post-placement adjustment, according to some embodiments of the present disclosure;

FIG. 40 illustrates yet another smart implant that is able to expand to account for post-operative settling and relaxation of soft tissue, according to some embodiments of the present disclosure;

FIG. 41 illustrates yet another smart implant that is also able to expand to account for post-operative settling and relaxation of soft tissue and includes sensing and stimulation electrodes, according to some embodiments of the present disclosure;

FIG. 42 illustrates a smart solution framework chart for pseudarthrosis following lumbar interbody fusion, according to some embodiments of the present disclosure;

FIGS. 43-44 illustrate a smart implant with a conductive graft scaffold, according to some embodiments of the present disclosure;

FIG. 45 illustrates fusion progress, impedance, and dynamic loading sensed over time by a smart implant, according to some embodiments of the present disclosure;

FIG. 46 illustrates a smart solution framework chart for periprosthetic joint infection following TKA, according to some embodiments of the present disclosure;

FIG. 47 illustrates a smart solution framework chart for mechanical loosening of the implant following TKA, according to some embodiments of the present disclosure;

FIG. 48 illustrates a smart solution framework chart for mechanical loosening of the implant following THA, according to some embodiments of the present disclosure;

FIG. 49 illustrates a smart solution framework chart for dislocation of the implant following THA, according to some embodiments of the present disclosure;

FIG. 50 illustrates a smart solution framework for adjacent segment disease (ASD) following lumbar interbody fusion, according to some embodiments of the present disclosure;

FIG. 51 illustrates rods or screws inserted within a femoral neck of a patient, according to some embodiments of the present disclosure;

FIG. 52 illustrates rods or screws inserted within the femoral neck and is connected to a smart implant or is in contact with an external device, according to some embodiments of the present disclosure;

FIG. 53 illustrates a method performed by a musculoskeletal implant device, according to embodiments of the present disclosure;

FIG. 54 illustrates another method performed by a musculoskeletal implant device in a closed-loop system, according to embodiments of the present disclosure; and

FIG. 55 illustrates yet another method performed by a musculoskeletal implant device in an open-loop system, according to embodiments of the present disclosure.

### DETAILED DESCRIPTION

The following discussion is presented to enable a person skilled in the art to make and use embodiments of the present disclosure. Various modifications to the illustrated embodiments will be readily apparent to those skilled in the art, and the principles herein can be applied to other embodiments and applications without departing from embodiments of the present disclosure. Thus, the embodiments are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of the embodiments. Skilled artisans will recognize the examples provided herein have many useful alternatives and fall within the scope of the embodiments.

One opportunity area is the enhancement of surgical execution. However, there remains a lack of information from the implants that are placed within a patient.

An opportunity exists to create smart musculoskeletal implants that provide feedback to the surgeon to ensure safe and accurate placement and optimal loading. Another intraoperative challenge is that the alignment and loading conditions of the construct are constantly changing throughout the procedure as anatomy is manipulated, modified, and instrumented with implants. For these issues, an opportunity exists to create smart musculoskeletal implants that enable a surgeon, surgery assisting system, or automated surgical system to optimize the alignment and loading of each implant and the global anatomical construct after all implants are placed.

Possible advantages that may be provided by one or more of the embodiments disclosed herein may include: providing feedback to the surgeon to ensure safe and accurate placement and optimal loading of implants; enabling the surgeon optimize the alignment and loading of each implant and the global anatomical construct after all implants are placed; providing feedback to the clinical team regarding the progress of bone growth and actively contributes to the acceleration of bone growth in a therapeutic capacity; providing the clinical team with feedback on the changes in positioning, orientation, and loading of implant components after surgery and enable the surgeon to noninvasively adjust the position, orientation, or state of implant components to correct for anatomic alignment or loading conditions that deviate from the surgical goal; enabling clinician to monitor, detect, or diagnose the onset of postoperative complications; providing non-invasive treatment options that can be remotely activated by the clinician or autonomously activated in a closed-loop system; and enabling entirely new diagnostic methods, surgical procedures, and less-invasive treatments that were previously unimaginable or impossible with traditional, purely mechanical implants.

Another opportunity area is the improvement of surgical outcomes, healing, and recovery. Minimally invasive surgical techniques have reduced hospital stays and improved the healing time of the surgical site, however, there have been limited advancements in improving the probability and rate of bone growth. For many procedures, success is dependent on some form of bone growth, which may include union of a fracture site in trauma repair, bridging between spinal vertebrae in spinal fusion, or in-growth and on-growth in cementless joint arthroplasty. An opportunity exists to create smart musculoskeletal implants that provide feedback to a clinical team regarding the progress of bone growth and actively contribute to the acceleration of bone growth in a therapeutic capacity. Even if the bone successfully fuses, the outcome of the procedure can be compromised. In spinal procedures, failed functional outcomes can often related to insufficient neural decompression, which can be caused by a loss of disk height or change in lordotic alignment due to changing loading conditions in the perioperative period. The patient may be properly aligned on the surgical table, but biomechanical loading of the implant construct changes drastically when the patient is transferred to the recovery bed and stands for the first time. These changing conditions can cause subsidence of the implants that result in compression of the neural structures and result in pain. Additionally, loss of lordotic correction can disrupt the patient's global alignment and cause increased stresses and degradation of adjacent vertebral levels as well as non-adjacent joints, such as the hip and knee. An opportunity exists to create a smart musculoskeletal implant that may provides the clinical team with feedback on the changes in positioning, orientation, and loading of implant components during or after surgery and enable the surgeon to noninvasively adjust the position, orientation, shape, or state of implant components to correct for anatomic alignment or loading conditions that deviate from the surgical goal.

Another opportunity area is the prevention and treatment of surgical complications. Common complications following musculoskeletal surgery include pain, infection, bone growth issues (e.g. pseudarthrosis), instability (e.g. dislocation), adjacent segment disease, and, in the case of tumor resections, cancer recurrence, among others. An opportunity exists to create smart musculoskeletal implants that monitor, detect, or diagnose the onset of postoperative complications. Additionally, an opportunity exists to create smart musculoskeletal implants that provide non-invasive treatment options that can be remotely activated by the clinician or autonomously activated in a closed-loop system.

Finally, an opportunity exists to create smart musculoskeletal implants that enable diagnostic methods, surgical procedures, and less-invasive treatments that were previously unimaginable or impossible with traditional, purely mechanical implants. Smart implants provide the opportunity to break through our current technological limits and enter a new territory of clinical effectiveness. With the integration of electronics, miniature actuators, and connectivity, implants can be capable of capturing data and delivering treatments like never before. This disclosure describes a number of novel embodiments and methods of use of smart musculoskeletal implants that provide clinical impact to the previously described opportunity areas.

Below embodiments to different types of musculoskeletal implants are provided. However, it should be noted that aspects and embodiments of certain musculoskeletal implants can be used in other types of musculoskeletal implants discussed herein.

A smart musculoskeletal implant (also referred to as an implant and/or smart implant herein) is an implantable medical device with control and communication capabilities designed to promote healing, prevent complications, and/or monitor surgical outcomes in patients with musculoskeletal disorders. The general embodiment of a smart musculoskeletal implant (i.e., smart implant) includes a control and communication module that is assembled to, integrated with, or part of the musculoskeletal implant, which is further described below.

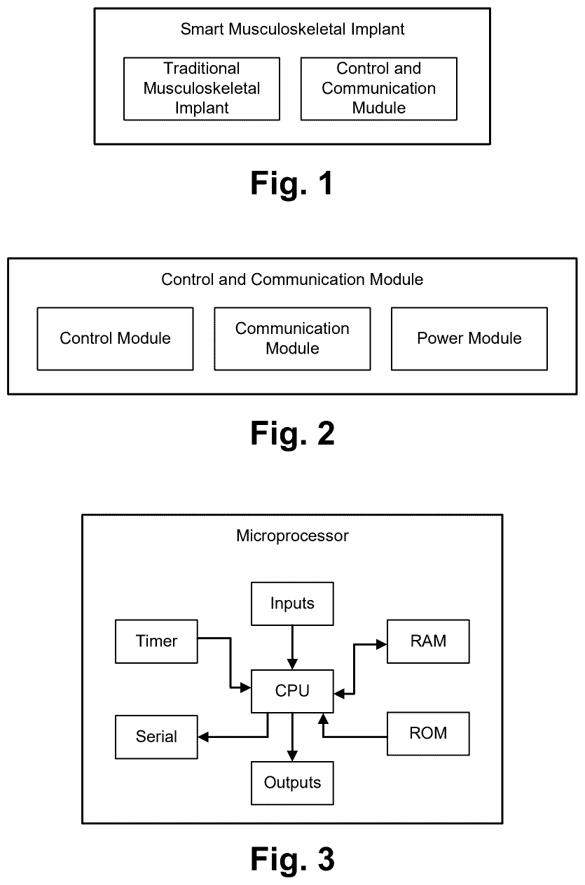

FIG. 1 illustrates an example of a smart musculoskeletal implant that includes a traditional musculoskeletal implant and a control and communication module, according to some embodiments of the present disclosure.

Musculoskeletal implants include any implant designed for musculoskeletal surgery, including spine, trauma, and joint procedures, which can be made ‘smart’ by the addition of an electronics module.

### Spinal Implants:

For spines, these traditional musculoskeletal implants can be used in anterior cervical, posterior cervical, anterior lumbar, lateral lumbar, posterior lumbar, sacroiliac, vertebral augmentation, deformity correction, growth modulation, and vertebral body replacement procedures. Implants for anterior cervical procedures include static interbody spacers, expandable interbody spacers, interbody spacers with integrated fixation plates, fixation anchors, fixation screws, anterior cervical plates, transfacet screws, and artificial disks. Implants for posterior cervical procedures include fixation screws, rods, locking caps, revision connectors, laminoplasty plates, and occipital plates. Anterior lumbar implants include static interbody spacers, expandable interbody spacers, interbody spacers with integrated fixation, fixation anchors, fixation screws, anterior plates, spondylolisthesis reduction systems, and artificial disks. Lateral lumbar implants include static interbody spacers, expandable interbody spacers, interbody spacers with integrated fixation, fixation anchors, fixation screws, and lateral plates. Posterior lumbar interbody spacers include static interbody spacers, expandable interbody spacers, articulating interbody spacers, articulating expandable interbody spacers, and lateral expanding interbody spacers. Posterior fixation implants include fixation screws, locking caps, rods, towers, modular screw shanks, modular screw heads, extendable growing rods, sublaminar bands, anterior staples, interspinous fixation systems, and semi-rigid transitional stabilization systems. Sacroiliac implants include sacroiliac joint fusion screws, sacroiliac joint fusion spacers, and S2-alar screws. Vertebral augmentation implants include vertebral compression fracture systems. Growth modulation implants include tethers, fixation screws, locking caps, and staples. Vertebral body replacement implants include static corpectomy spacers, expandable corpectomy spacers, corpectomy spacers with integrated fixation, fixation screws, and fixation anchors.

### Trauma Implants:

Traditional musculoskeletal implants in trauma surgery include general implants as well as implants specific to anatomy, including clavicle, proximal humerus, elbow, hand and wrist, pelvis, hip, femur, tibia, foot and ankle, and craniomaxillofacial. General trauma implants include large fragment plates, mini fragment plates, monoaxial locking plates, locking screws, non-locking screws, and external fixator pins. Clavicle implants include contoured clavicle plates. Proximal humerus implants include proximal humerus contoured plates, proximal humeral nails, and humeral nails. Elbow implants include variable angle locking plates, contoured elbow plates, triad plates, and radial head replacement components. Hand and wrist implants include distal radius plates, distal ulna plates, phalangeal plates, metacarpal plates, wrist arthrodesis plates, finger joint arthroplasty implants, and tendon rods. Pelvis implants include pelvic plates, acetabulum plates, and pelvic compression screws. Hip implants include trochanteric nails, intermediate trochanteric nails, long intertrochanteric nails, antegrade femoral nails, intertrochanteric compression screws, lag screws, and compression screws. Femur implants include retrograde intramedullary nails, distal femur plates, and knee arthrodesis nails. Tibia implants include tibia intramedullary nails, distal tibia plates, and proximal tibia plates. Foot and ankle implants include osteotomy wedges, distal fibula plates, neuropathic osteoarthropathy intramedullary nails, staples, phalangeal plates, metatarsal plates, triplanar plates, and syndesmosis devices. Craniomaxillofacial implants include craniotomy plates, orbital floor plates, mandible plates, and fixation screws.

### Joint Implants

Traditional joint arthroplasty implants can be used in cemented and cementless knee arthroplasty, cemented and cementless hip arthroplasty, shoulder arthroplasty, elbow arthroplasty, ankle arthroplasty, and wrist arthroplasty. Implants for knee arthroplasty include total knee replacement implants, including femoral components, patella components, tibial inserts, and tibial trays, revision total knee implants, including tibial extension components, femoral extension components, and augment components, and unicompartmental knee implants. Implants for hip arthroplasty include total hip replacement implants, including acetabular components, acetabular liners, femoral heads, and femoral stems, revision total hip implants, including femoral stem extension components, pelvic augment components, cerclage wires, and acetabular shell fixation screws, and partial hip implants, including femoral head resurfacing components. Implants for shoulder arthroplasty include total shoulder replacement implants, including humeral head components, humeral stems, and glenoid fossa components, reverse total shoulder replacement implants, including humeral cups, humeral stems, and glenoid sphere components, as well as shoulder hemiarthroplasty components. Elbow arthroplasty implants include humeral components, bearing liners, ulnar components, and hinge pins. Ankle arthroplasty implants include talar components, bearings, spacers, and tibial components. Total wrist arthroplasty implants include radial components, carpal ball components, and carpal plate components.

### Control and Communication Module

FIG. 2 illustrates an example of a control and communication module that includes a control subsystem, communication subsystem, and power subsystem, according to some embodiments of the present disclosure.

The control and communication module can be considered the element which makes the system ‘smart’. The control and communication module includes a control subsystem, communication subsystem, and power subsystem. These subsystems may be combined into a single subsystem, but for clarity each subsystem is described independently below.

### Control Subsystem:

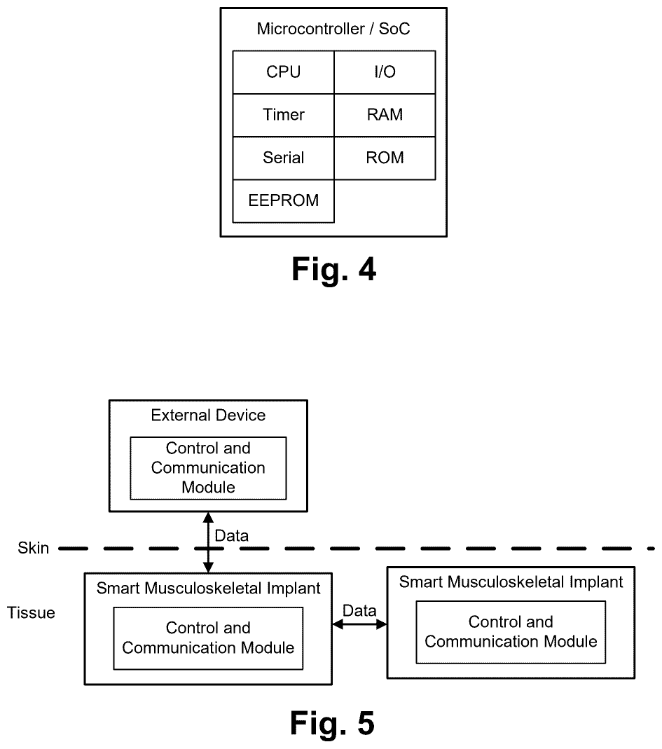

FIGS. 3-4 illustrate a control subsystem, according to some embodiments of the present disclosure.

In some embodiments, the control subsystem is responsible for control of the smart implant system, which may include the communication subsystem and other subsystems, such as sensors, actuators, and/or power, which are further described below. The control subsystem includes central processing unit (CPU), which is responsible for processing inputs, interpreting commands, running control algorithms, and processing outputs to peripheral devices. The control subsystem may include a microprocessor unit (MPU), microcontroller unit (MCU), or system-on-a-chip (SoC). In a microprocessor, the CPU is dedicated chip which communicates with other chips with dedicated functions, which may include timers, a serial interface, random access memory (RAM), read-only memory (ROM), as well as inputs and outputs. In a microcontroller or SoC, these subsystems are all integrated onto a single integrated circuit (IC). Greater miniaturization, processing speed, efficiency, design flexibility, and long-term cost can be achieved with SoCs and alternate architectures, including application-specific integrated circuits (ASICs) and field-programmable gate arrays (FPGAs). In these architectures, control subsystems as well as other subsystems and circuitry are integrated onto a single integrated circuit.

### Communication Subsystem:

FIG. 5 illustrates a smart musculoskeletal implant implanted in a patient's body and is communicatively coupled with a non-implanted external device and another smart musculoskeletal implant implanted in the patient's body, according to some embodiments of the present disclosure.

The communication subsystem is responsible for transmitting (Tx), receiving (Rx), or transmitting and receiving (Tx/Rx) data to a separate device. The separate device may include one or more other smart implants as well as the non-implanted, external device including a smartphone, tablet, computer, or a dedicated controller device. Wireless communication methods include radio frequency (RF) methods, inductive methods, optical methods, electrical methods, and ultrasonic methods. The received data may include data or commands from other implants, data or commands from the external device, as well as over-the-air software/firmware updates. The transmitted data may include data or commands to other implants as well as data to the external device.

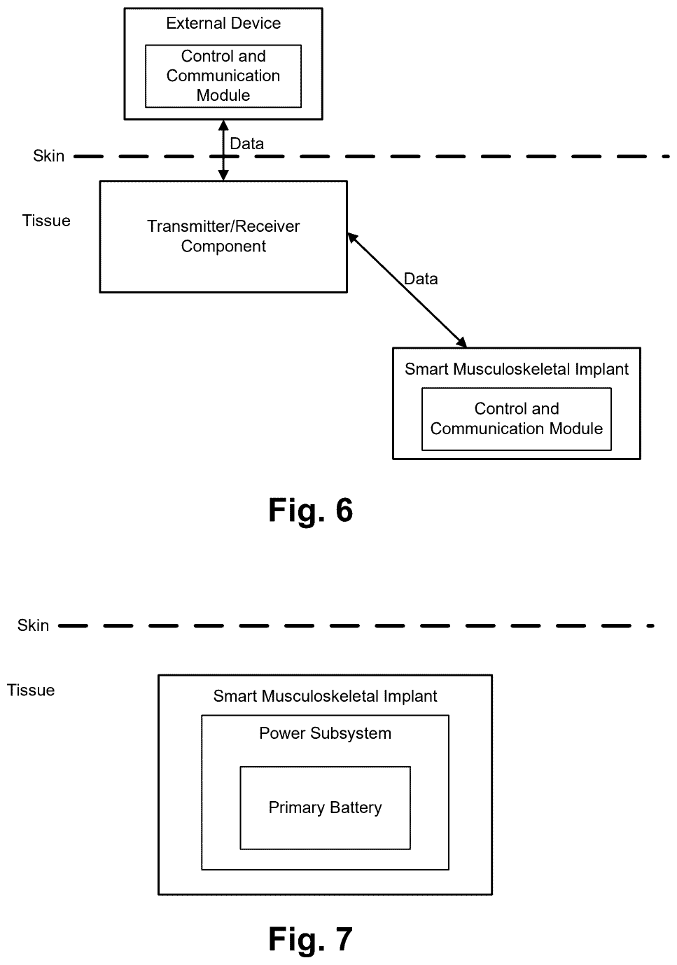

The transmitter/receiver component of the communication subsystem may be integrated within the smart musculoskeletal implant assembly or may be physically separate, but communicatively connected component. The physically separate, but communicatively connected component may be communicatively connected to the smart musculoskeletal implant via a wire or may be wirelessly connected. Separation from assembly may be advantageous to enable the Tx/Rx component to be placed in closer proximity to the skin to improve transmission quality and efficiency and positioned in an anatomic location that improves usability for the patient, for example as shown in FIG. 6

FIG. 6 illustrates a smart musculoskeletal implant implanted in a patient's body and includes a physically separate but communicatively connected component that is also implanted in a patient's body which is used to communicate with a non-implanted external device, according to some embodiments of the present disclosure.

A preferred embodiment utilizes RF communication because it is the only method that does not require an external receiver device to be placed on or in close proximity to the implanted transmitter. RF communication can require some form of antenna to be part of or connected to the communication subsystem. Embedded RF antennas include ceramic chip antennas, microstrip patch antennas, and printed circuit board (PCB) trace antennas. PCB trace antenna configurations include the meandered dipole, folded dipole, dipole, or monopole. External RF antennas include wire antennas, lens antennas, array antennas, aperture antennas, reflector antennas, patch antennas, and microstrip antennas. The allowable RF communication frequency bands, regardless of antenna type, for implants may include 30-37.5 MHz, 401-457 MHz, 603-614 MHz, 902-928 MHz, 1395-1499 MHz, 2360-2400 MHz, and 2483-2500 MHz. Some preferred frequency bands for smart implants may include 402-405 MHz, 915 MHz, and 2.4 GHz. Standard protocols for these preferred bands include Bluetooth and Bluetooth Low Energy (BLE) at 2.4 GHz and medical implant communication system (MICS) at 402-405 MHz.

Other embodiments may include the following other communication methods, which require an external device to be placed on or adjacent to the skin. Inductive coupling relies on one or more implanted wire coil which must be closely aligned with an external coil placed over or on the skin. Resonance inductive coupling is more forgiving alignment and range requirements and relies on tuned wire coils or lumped element resonators to communicate encoded signals across the tissue layers to an external device. Optical methods involve encoding data into pulsed light signal which can be transferred through the skin layer and read through an external device, such as a wearable. Electrical methods involve encoding data into pulsed electrical signals which can be transmitted through the body's naturally conductive structures and interpreted with electrodes placed on the skin or via a wearable device, such as a smart watch. Ultrasonic methods use piezoelectric resonators which transmit ultrasonic waves through the tissue which can be read with an ultrasonic receiver on the skin.

### Power Subsystem

FIG. 7 illustrates a power subsystem within the smart musculoskeletal implant that is implanted in a patient's body, according to some embodiments of the present disclosure.

The power subsystem provides energy to enable operation of the smart implant. Configurations of the power subsystem include stored energy embodiments, harvested energy embodiments, wirelessly transferred energy embodiments, or a combinations thereof.

Stored energy embodiments use energy which is stored within an implanted module, such as a traditional electrochemical battery, solid state battery, or supercapacitor. Energy storage devices may be energized during manufacturing, prior implantation, and utilized until the available power is depleted (i.e. primary cell). Alternatively, the energy storage device may be recharged after implantation (i.e. rechargeable cell) through intentional wireless energy transfer mechanisms or energy harvesting from host environment. Methods of energy harvesting and wireless energy transfer are further discussed below.

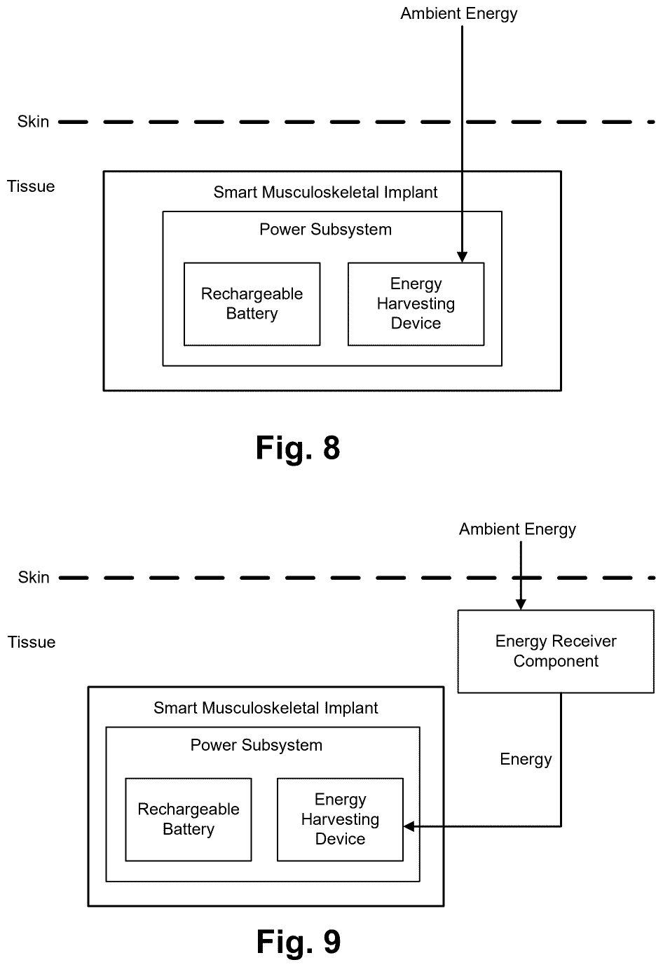

FIG. 8 illustrates a power subsystem of a smart musculoskeletal implant that includes an energy harvesting device and rechargeable battery that capture energy from an ambient environment, according to some embodiments of the present disclosure. In some embodiments, the power subsystem captures the energy from the ambient environment directly. In other embodiments, as shown in FIG. 9, an energy receiver component may capture the energy from the ambient environment and transfer the energy to the energy harvesting device.

FIG. 9 illustrates another power subsystem of a smart musculoskeletal implant that includes an energy harvesting device and rechargeable battery that capture energy from an energy receiver component that is physically separate from the smart musculoskeletal implant, according to some embodiments of the present disclosure.

The harvesting energy device captures energy from ambient energy that comes from the ambient environment and converts it to an electrical form which can be stored in the rechargeable batter and power the smart implant. The ambient environment includes the internal ambient environment of the body in which the implant is placed within, or the external ambient environment in which the patient lives. Examples of these ambient energy sources in the ambient environment include thermal energy, kinetic energy, and RF energy.

Energy harvesting devices that capture thermal energy utilize thermoelectric generators (TEGs), which consist of a junction of two dissimilar metals placed within a thermal gradient. The power output of a TEG is dependent on the scale of the temperature gradient, which may be enhanced by the addition of a heat sink. One embodiment of an implantable TEG involves capture of a temperature gradient between a heatsink contained within an insulated implant module and the external tissue.

In a preferred embodiment, placement of the TEG within or adjacent to a load-bearing joint, such as the knee or hip. For example, as heat is generated from the mechanical motion of the joint, a TEG placed within the femoral component or tibial spacer component of a knee arthroplasty smart implant construct captures the thermal energy and converts it to useable electrical energy.

Energy harvesting devices that capture kinetic energy convert motion, vibration, or forces into electrical energy. Kinetic energy harvesting methods include electromagnetic, piezoelectric, or triboelectric. Electromagnetic energy harvesting configurations include linear and rotary embodiments. Linear electromagnetic kinetic energy harvesters include a plunger which translates within a coil winding during kinetic motion, which includes an electromagnetic field and current within the coil. Rotary electromagnetic kinetic energy harvesters (i.e. generators) work similar, but instead the kinetic motion induces rotation of a rotor relative to a stator that induces an electromagnetic field and current within the coils. One embodiment of an implanted EM kinetic energy harvesting system may include the placement of a rotary harvester device within the joint of a hinged total knee implant, which can capture the rotational motion of the knee joint and convert it to useable electrical energy.

Piezoelectric generators rely on the properties of piezoelectric materials (e.g. quartz), which generate an electrical signal when a mechanical force is applied. The output of a piezoelectric kinetic energy harvesting system can be multiplied by stacking multiple piezoelectric generators. One embodiment of an implanted piezoelectric kinetic energy harvesting system may include one or more piezoelectric stacks within an interbody spacer implant, which can capture stress translated from the adjacent vertebral bodies and convert it to electrical energy. Triboelectric kinetic energy harvesting devices (e.g., a triboelectric nanogenerator (TENG)) utilize the triboelectric effect, which uses electric charge transfer between two objects when they contact or slide against each other. One embodiment of an implanted triboelectric system may include a structure assembled or 3D printed from two or more dissimilar materials that is placed within a dynamic joint such as the knee. In some embodiments of a TENG, the assembly includes four layers: a charge-generating layer, a charge-trapping layer, a charge-collecting layer, and a charge storage layer. In some embodiments, the dissimilar materials include electron donor materials and electron acceptor materials. The electron donor materials include, but are not limited to, aluminum, coper nylon, silver, and gold. The electron acceptor materials include, but are not limited to, polytetrafluoroethylene (PTFE), polydimethylsiloxane (PDMS), and polyimide. In the application of total knee arthroplasty, the tibial spacer component could be crafted from layers of alternating materials which minutely compress and slide generating electricity during motion of the knee joint.

Radiofrequency (RF) energy harvesting devices capture RF energy travelling through free space, which could include WiFi, Bluetooth, AM/FM radio, and other signals. RF harvesters work by capturing the transmitted signal with a receiving antenna and impedance matching network that is tuned to the frequency of the target signal (e.g. 2.4 GHz for WiFi). The captured signal is then rectified and boosted using a rectifier and voltage multiplier circuit, where it is converted to a DC voltage that can be used or stored. One embodiment of an implanted RF energy harvesting system that includes a smart spinal fixation rod that has a dipole antenna integrated along its length that captures ambient WiFi and Bluetooth energy at 2.4 GHz, a circuit to convert the energy to DC, a battery that stores the energy, a strain gauge that measures loading on the rod, and a control system that transmits the recorded strain measurement wirelessly with the same integrated antenna system.

An alternative to passively harvesting ambient energy is to wirelessly transmit energy from an external device to an implanted receiver (e.g., wireless energy transfer device). Wireless energy transfer system configurations include inductive coupling, resonant inductive coupling, capacitive coupling, magneto dynamic coupling, radiofrequency transmission, and optical transmission. The working principles of some of the described embodiments herein are identical or similar to the previously described wireless communication embodiments, however, instead of transmitting or receiving a data signal, the implant converts the received signals into useable energy. Additionally, the wireless energy transfer mechanism can be configured to enable a bidirectional data link while the implant receives power from the external source.

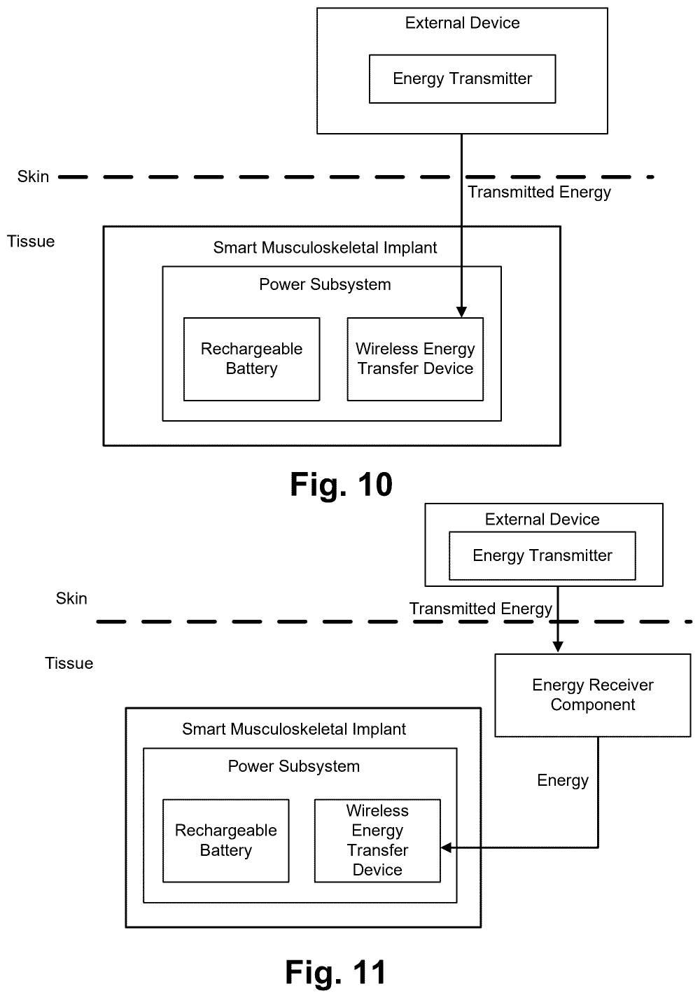

FIG. 10 illustrates a power subsystem of the smart musculoskeletal implant that is configured to receive transmitted energy from an external device, according to some embodiments of the present disclosure.

FIG. 11 illustrates another power subsystem of the smart musculoskeletal implant that is configured to receive transmitted energy from an energy receiver component that is physically separate from the smart musculoskeletal implant, according to some embodiments of the present disclosure.

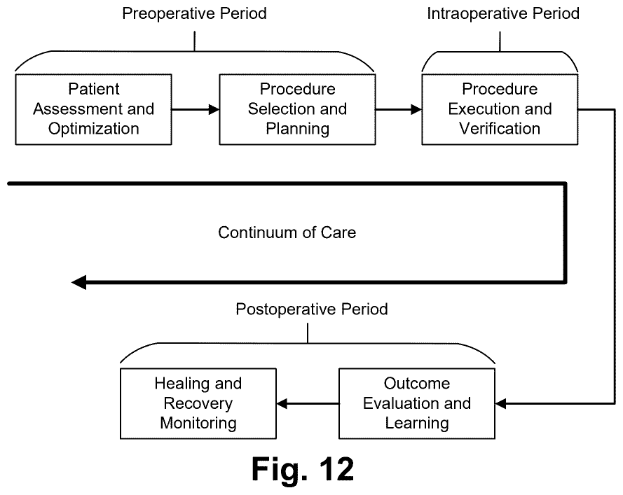

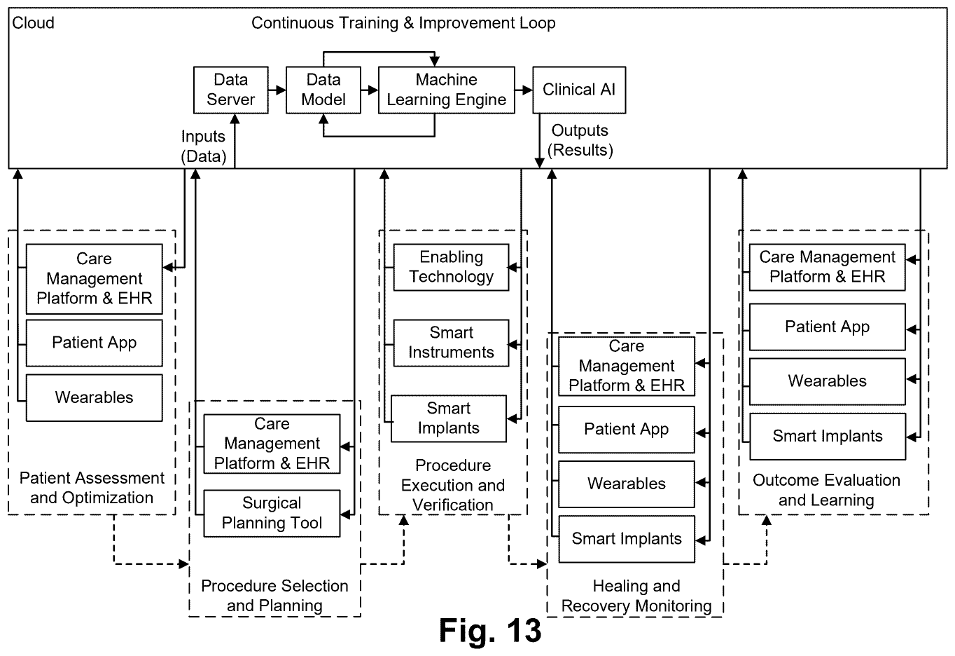

Embodiments discussed herein can be used in many applications across the continuum of care. FIG. 12 illustrates periods throughout the continuum of care in which embodiments can be used.

### Connected Care System:

FIG. 13 illustrates a connected care system, according to some embodiments of the present disclosure.

Smart implants can be utilized within a connected care system to add value across the continuum of care. The continuum of care encompasses a patient's journey from the onset of symptoms to treatment to longitudinal evaluation and can be divided into five stages: patient assessment and optimization, procedure selection and planning, procedure execution and verification, healing and recovery monitoring, and outcome evaluation and learning.

Through the connected care system with other edge devices, software, and machine learning, smart implants can directly (e.g., via therapy delivery) or indirectly (e.g., via data collection) add value at each stage in the continuum of care. This connected care system includes a care management platform with electronic health record (EHR) integration, patient engagement app, wearable devices, intelligent surgical planning tools, enabling technologies, smart instruments, and smart implants as well as a cloud connectivity infrastructure. Each element in the system can feed data to a cloud, which can be used to continuously train and improve machine learning models. These models produce clinical artificial intelligence (AI) that can be sent to each edge device and interface to be utilized by the clinician, software, or device. This actionable clinical AI includes treatment recommendations, surgical plans, diagnostics, risk prediction, and alerts. The AI may also include closed-loop or surgeon-in-the-loop control algorithms for the smart implant devices that provide intraoperative features, such as self-optimizing loading adjustment, or other postoperative features, such as autonomous detection and treatment of periprosthetic joint infection.

### Intraoperative Connectivity—Operating Room

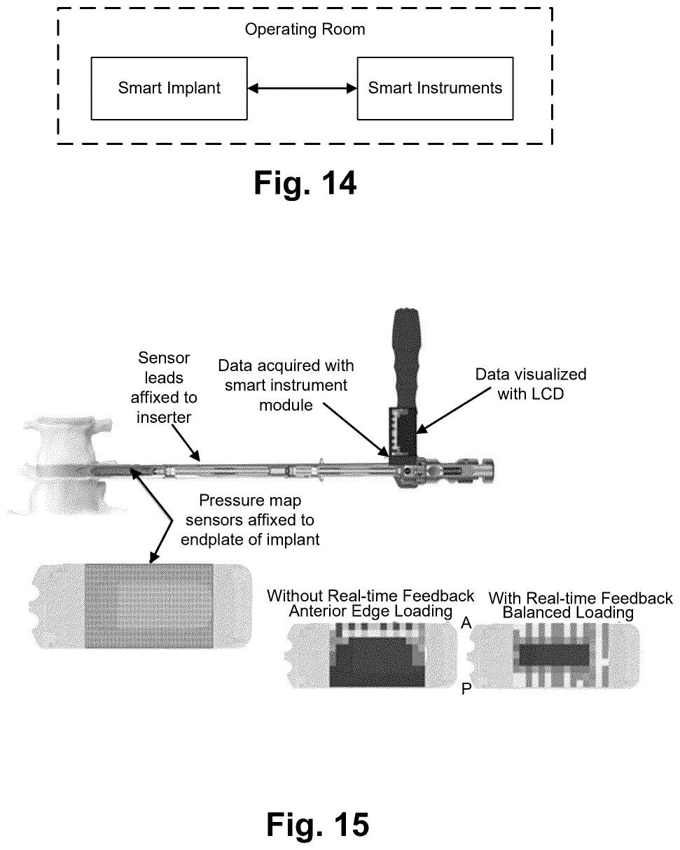

FIG. 14 illustrates an operating room that includes a smart implant and smart instruments that are communicatively connected to each other, according to some embodiments of the present disclosure.

FIG. 15 illustrates a smart implant and smart instrument that is configured to provide real-time (or near real-time) feedback to a surgeon, according to some embodiments of the present disclosure.

Some level of wired or wireless connectivity is required for a smart implant to provide feedback (e.g, sensor information received from at least one sensor connected to the smart implant) to the surgeon in the operating room. This connectivity primarily functions for data transmission, however, it may also be utilized to power the smart implant during surgery. One embodiment of intraoperative connectivity consists of a smart implant connected to a smart instrument, which possesses an interface to enable unidirectional or bidirectional communication between the surgeon and smart implant.

With reference to FIG. 15, in some embodiments, smart implant (e.g., smart interbody spacer) is connected to a smart instrument (e.g., smart interbody inserter). The smart implant can measure or otherwise obtain sensor information (e.g., loading) from sensors connected to the smart implant (e.g., endplates connected to or integrated in the smart implant) during insertion, expansion, and/or compression of the smart implant. The sensor information can be transmitted from the smart implant to an external device (e.g., a device on the smart instrument). The sensor information can be displayed on the external device (e.g., loading distribution can be displayed on a screen (e.g., LCD screen)). The surgeon can then use the displayed sensor information to determine a modification of a feature associated with the smart implant (e.g., positioning and/or expansion of the smart implant) to optimize loading distribution across the implant while achieving decompression and lordotic alignment goals. Then the surgeon can modify the feature associated with the smart implant based on the sensor information.

While some embodiments herein include data transmitted through wires (e.g., sensor leads affixed to the smart insert), it should be noted that wireless data transmission may be used as an alternative or additional method of data transmission herein.

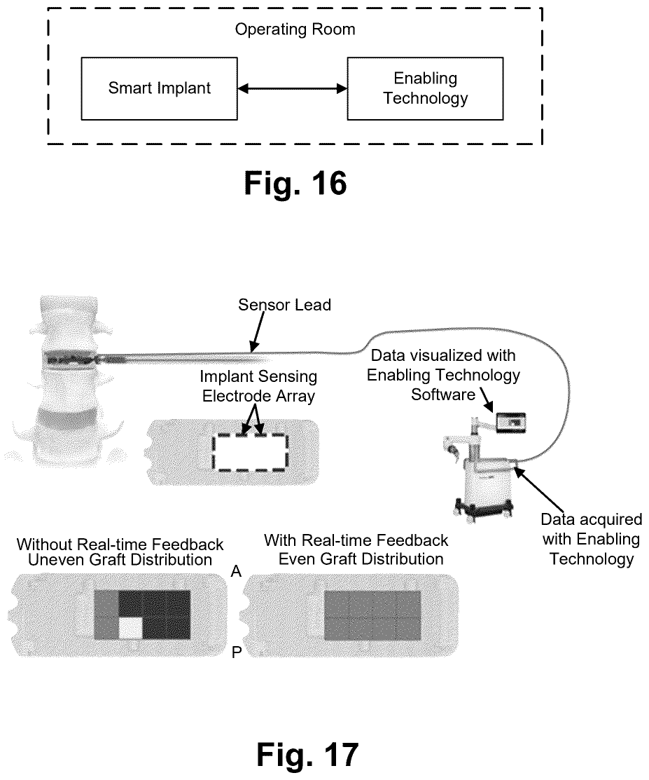

FIG. 16 illustrates another operating room that includes a smart implant and an enabling technology, according to some embodiments of the present disclosure.

FIG. 17 illustrates another smart implant and another smart instrument that is configured to provide real-time (or near real-time) feedback to enabling technology, according to some embodiments of the present disclosure.

Another embodiment of intraoperative connectivity includes the smart implant connected to the enabling technology. The enabling technology may include an imaging system, navigation system, robotic system, table system, standalone computer, or combination thereof. In the example of FIG. 17, the smart implant is a smart interbody spacer configured to measure the density of graft tissue placed within its graft window during a backfilling step and displays a distribution heatmap of the inserted tissue density over a rendered implant model and medical image on a navigation system of the enabling technology. The navigation system may enable the surgeon to objectively assess the volume, density, and location of graft placed during the backfilling step.

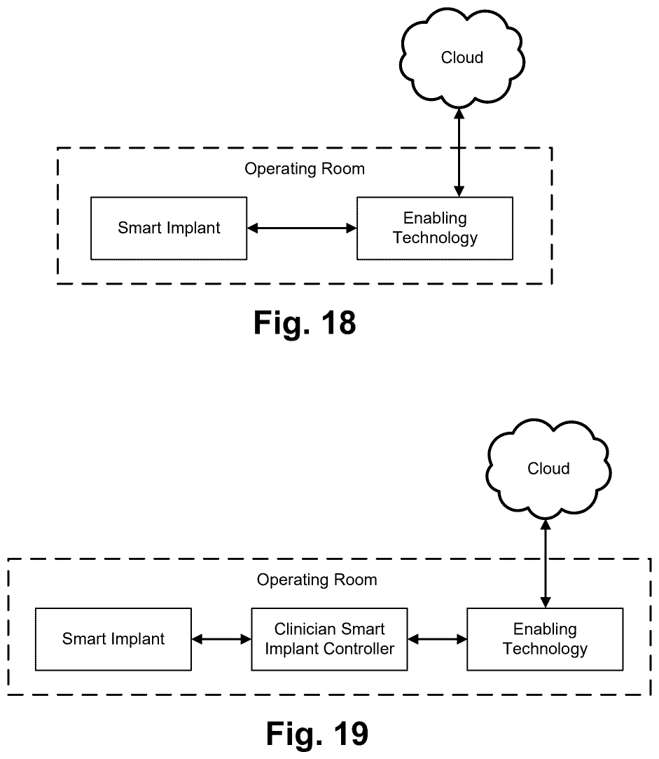

FIG. 18 illustrates an embodiment of intraoperative connectivity that includes a smart implant connected to an enabling technology, which is connected to a cloud, according to some embodiments of the present disclosure.

In some embodiments, the cloud includes a data server, machine learning engine, and/or artificial intelligence. Connection to the cloud enables data to be fed to the cloud, processed with a complex algorithm(s), and used to provide surgical recommendations or commands to the surgeon and edge devices (i.e. smart implant or enabling technology).

An example of this embodiment is a smart spine fixation device which contains a bi-stable or variable stiffness mechanism that can be wirelessly actuated via an enabling technology, such as a smart table. In this example, the fixation device can be placed minimally invasively and all corrections can be performed non-invasively with the assistance of a smart table that can change shape (e.g. adjust lordotic angle) and apply directed forces with actuated bolsters. After satisfactory manipulations are made to the patient anatomy via the smart table, the implant mechanism can be wirelessly locked to enable a rigid corrected construct. In addition to surgeon-commanded manipulations, the system could be configured with surgeon-in-the-loop (feedback provided to a device for surgeon analysis) or fully autonomous control (feedback provided to an external device for processing and determination of modification instructions or processed within the smart implant and determined within the smart implant what feature(s) to modify) such that the loading and alignment of the patient anatomy are analyzed in real-time, compared with respect to the surgical plan, and modified via autonomous manipulations of the smart table elements. Such an embodiment also enables additional non-invasive corrections and adjustments postoperatively, as needed, to account for changes in postoperative loading conditions, soft tissue relaxation, and subsidence.

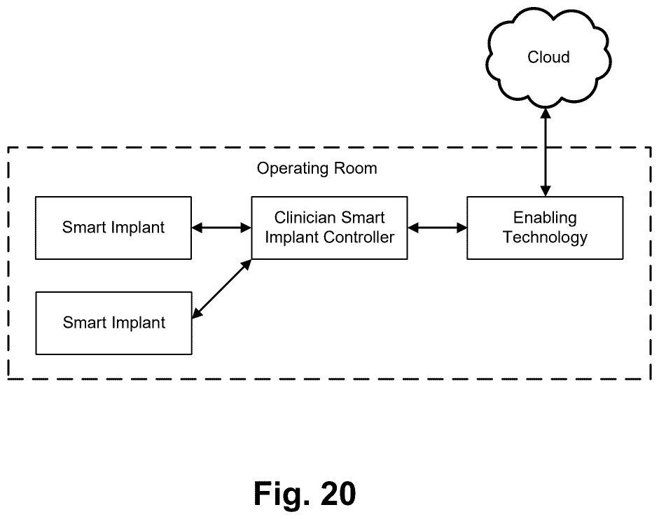

FIG. 19 illustrates another embodiment of intraoperative connectivity that includes a smart implant connected to an enabling technology via a smart implant controller device (e.g., a clinician smart implant controller), according to some embodiments of the present disclosure.

A dedicated smart implant controller device may be advantageous in scenarios where a smart implant is used with an enabling technology system, such as a legacy system, which does not have integrated connectivity subsystems capable of communicating with and/or powering the smart implant. An example of this embodiment includes a smart implant controller in the form of a wearable. The wearable may be adhered to the patient in the general location of the surgical region on the opposite side of the patient from the surgical access corridor to not disrupt the surgical workflow. This controller communicates with and provides power to the smart implant during surgery. The controller functions as a communication bridge between the smart implant and, for example, a legacy robotic system, which does not have integrated implant communication capabilities.

FIG. 20 illustrates yet another embodiment of intraoperative connectivity that includes two smart implants connected to an enabling technology via a smart implant controller device (e.g., a clinician smart implant controller), according to some embodiments of the present disclosure.

In the embodiments of FIG. 20, the clinician smart implant controller (or other previously described connected device) can communicate with two or more independent smart implants. An example of this embodiment includes two smart interbody spacers capable of load sensing placed at adjacent levels, which may both transmit loading data (simultaneously or sequentially) to the controller, which forwards the data to the enabling technology. Such a system could be advantageous for visualizing the loading data on a navigation screen, enabling the surgeon to optimize and balance loading across both interbody levels during a posterior fixation stage of the procedure.

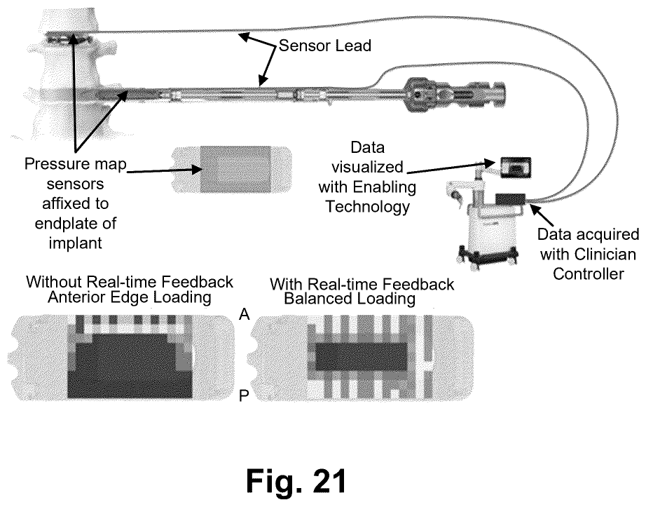

FIG. 21 illustrates a smart implant configured to communicate sensor information to an enabling technology via a controller (e.g., clinician controller), according to some embodiments of the present disclosure.

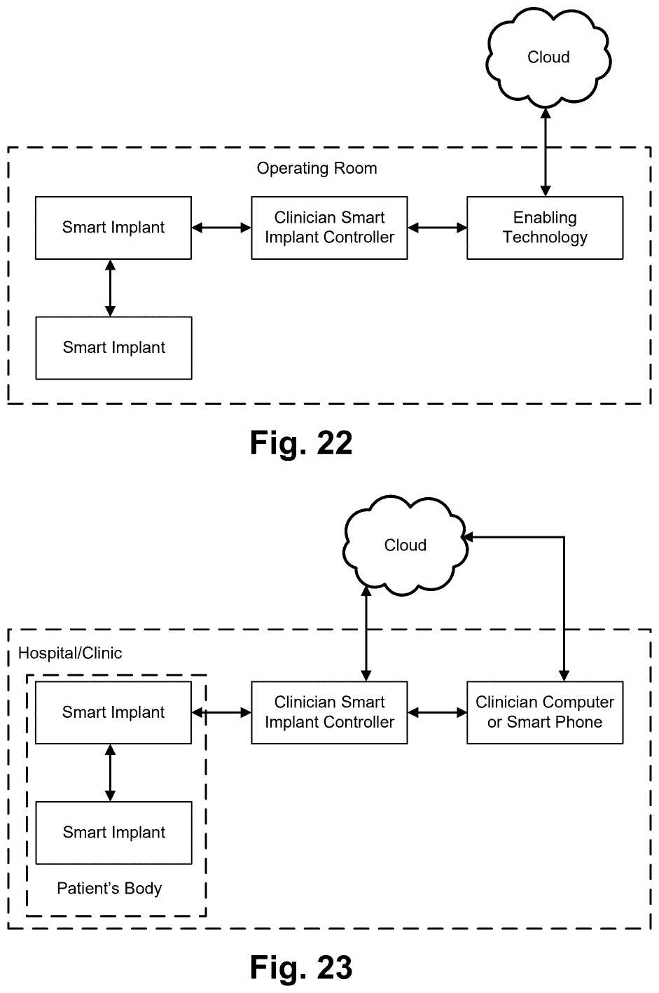

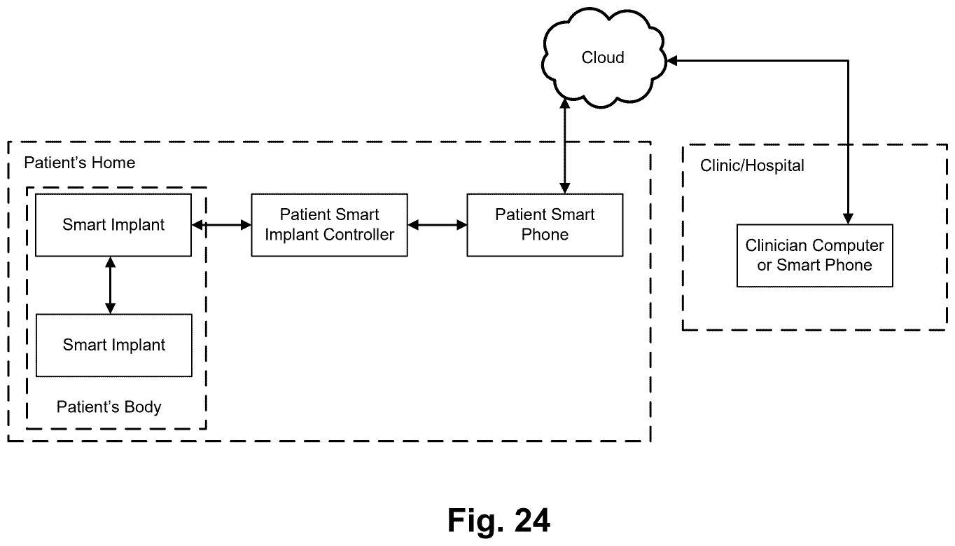

FIG. 22 illustrates yet another embodiment of intraoperative connectivity that includes a primary smart implant and a secondary smart implant connected to an enabling technology via a smart implant controller device (e.g., a clinician smart implant controller), according to some embodiments of the present disclosure.

In some embodiments, one or more secondary smart implants communicate with a primary smart implant, which serves as a bridge between the controller and all secondary smart implants. An example of this embodiment is a construct comprised of two smart rods and a smart interbody spacer, where one of the rods serves as the primary smart implant and the other rod and interbody spacer serve as secondary smart implants. Such a configuration may be advantageous in scenarios where the deepest smart implant (e.g. smart interbody) cannot effectively communicate with the external controller due to the amount of tissue between the devices. Particularly in patients with high body mass index (BMI), the distances of the wireless communication segments between the smart interbody and smart rod and smart rod and external device may be considerably less than the direct distance between the smart interbody and external device, making this configuration more effective and efficient.

### Postoperative Connectivity—Hospital & Clinic

FIG. 23 illustrates a hospital/clinic environment where a smart implant provides feedback to a clinician, according to some embodiments of the present disclosure.

Wireless connectivity may be required for a smart implant to provide feedback to the clinician (i.e. surgeon, physician assistant, nurse, etc.) in the hospital or clinic. This connectivity primarily functions for data transmission, however, it may also be utilized to power or charge the smart implant. One embodiment of intra-clinic or intra-hospital connectivity includes a smart implant that possesses an interface to enable unidirectional or bidirectional communication between the clinician smart implant controller and smart implant. An example of this embodiment is a smart fixation rod that receives power from and sends and receives data to/from a smart pad placed behind the patient in the hospital bed. The smart pad is of flexible construction and includes one or more RF antennas to send and receive data and send power. In some preferred embodiments, the RF frequencies include 402-405 MHz (MICS), 915 MHz, and 2.4 GHz (Bluetooth), or a combination thereof. The smart mat may be powered by a wired connection to a wall outlet or by a rechargeable battery. The smart mat may also be connected to the hospital internet and/or cloud via a wired ethernet connection or by a wireless WiFi connection. In this example, the smart fixation rod may monitor and report information regarding stress or strain of the implant, temperature of the wound, signs of biofilm growth and infection, and more. The smart fixation rod may also deliver bone growth stimulation treatment, nerve pain stimulation treatment, or biofilm eradication treatment via an autonomous or wirelessly activated control system. Further, the smart fixation rod may serve as an intermediate power and communication link between a contralateral smart fixation rod and/or a smart interbody spacer.

### Postoperative Connectivity—Patient's Home

FIG. 24 illustrates a patient's home environment where a smart implant provides sensor information to a clinician via a patient smart implant controller and/or a patient smart phone, according to some embodiments of the present disclosure. It should be noted that while a smart phone is discussed, this may include any device (e.g., computer, tablet, etc.) that is capable of receiving sensor information from the smart implant and/or a patient smart implant controller and relaying said information to a cloud.

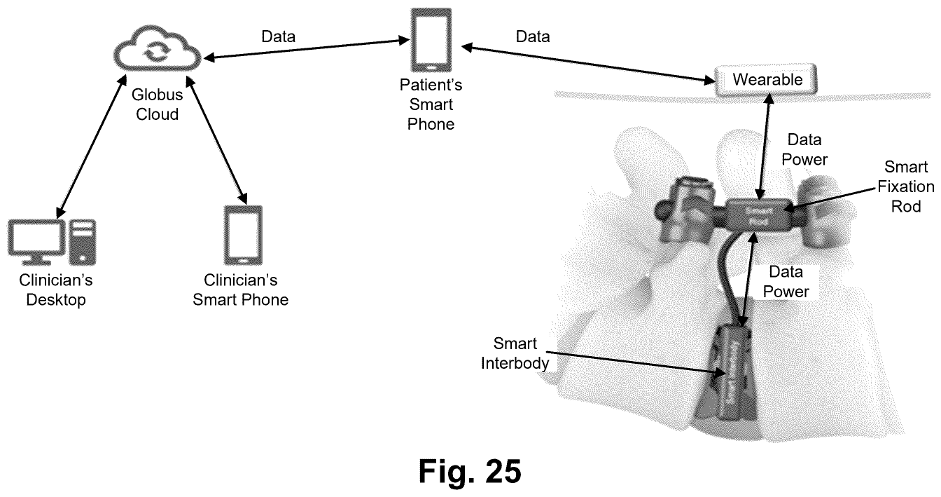

FIG. 25 illustrates a smart implant (e.g., smart interbody) connected to another smart implant (e.g., a smart fixation rod) that provides the smart implant's and the another smart implant's data to a wearable for providing feedback to a clinician, according to embodiments of the present disclosure.

Wireless connectivity may be required for a smart implant to provide feedback to the clinician (i.e. surgeon, physician assistant, nurse, etc.) when the patient returns home. This connectivity primarily functions for data transmission, however, it may also be utilized to power or charge the smart implant. One embodiment of intra-home connectivity includes a smart implant that possesses an interface to enable unidirectional or bidirectional communication between the clinician smart implant controller and implant. An example of this embodiment is a smart fixation rod that receives power from and sends and receives data to/from a smart pad placed behind the patient in their bed or preferred seating location. The smart pad is of flexible construction and includes one or more RF antennas to send and receive data and send power. In some preferred embodiments, the RF frequencies include 402-405 MHz (MICS), 915 MHz, and 2.4 GHz (Bluetooth), or a combination of these. The smart mat may be powered by a wired connection to a wall outlet or by a rechargeable battery. The smart mat may also be connected to the home internet and cloud via a wired ethernet connection, wireless WiFi connection with the home router, or wireless Bluetooth connection with the patient's smart phone. In this example, the smart fixation rod may monitor and report information regarding stress or strain of the implant, temperature of the wound, signs of biofilm growth and infection, and more. The smart fixation rod may also deliver bone growth stimulation treatment, nerve pain stimulation treatment, or biofilm eradication treatment via an autonomous or wirelessly activated control system.

Further, the smart fixation rod may serve as an intermediate power and communication link between a contralateral smart fixation rod and/or a smart interbody spacer.

Intraoperative Applications—Smart Implants that may Enhance Surgical Execution

This section further describes embodiments of smart musculoskeletal implants designed to enhance the execution of surgical procedures. These embodiments include smart implants that may provide a surgeon or a robotic system/enabling technology with feedback to ensure safe and accurate placement and optimal loading of the implants as well as enable the surgeon to optimize the alignment and loading of each implant and global anatomic construct during and after placement of all implants. In some embodiments, the proposed smart implant are not intended to be used as isolated tools, but rather elements of a connected surgical system that was previously described.

### Implant Placement Feedback

Safe and accurate placement of musculoskeletal implants is critical to achieve acceptable treatment outcomes. In some musculoskeletal surgeries, the placement of the implant is dictated by the surgical access corridor, tissue resection, and or bone prep. In these scenarios, such as in total knee arthroplasty (TKA), the implant is following the prepared path which places more emphasis on the preparation steps than the implant placement step. In other scenarios, such as the placement of self-drilling, self-tapping screws, the implant itself may be creating or contributing to the creation of the placement pathway. In these cases, the implant must be guided and inserted with extreme care. Despite advances in imaging, navigation, robotics, and neuromonitoring, there remains a gap in feedback to discern when the implant deviates from the intended pathway and is at risk of damaging sensitive tissue structures, such as the spinal cord or major vessels. In scenarios where the placement of the implant can cause damage to the surrounding tissues, there exists an opportunity for smart implants to provide feedback to the surgeon or robotic system that guides implant placement.

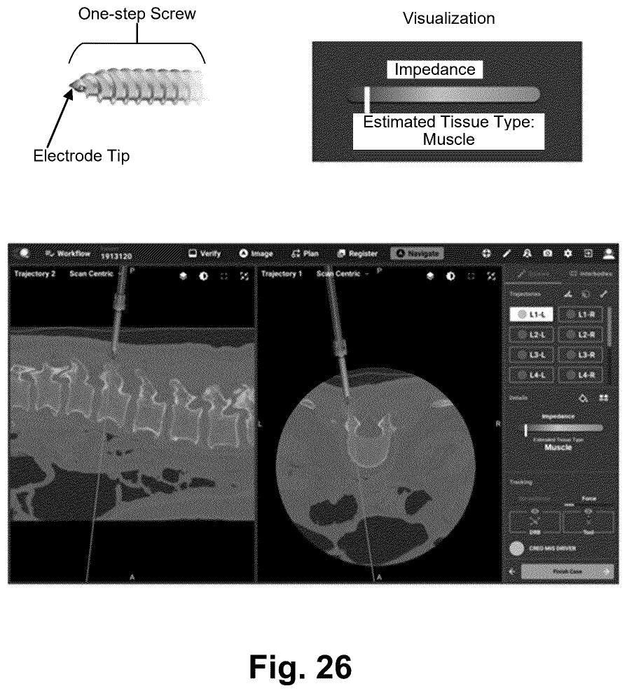

FIG. 26 illustrates a smart musculoskeletal implant that includes a screw capable of sensing the type of tissue that is in contract with, or in proximity to, the screw, according to some embodiments of the present disclosure.

One embodiment of a smart musculoskeletal implant that aids in the guidance of its placement includes a screw (e.g. pedicle screw in spine, lag screw in trauma, acetabular screw in joints) that is capable of sensing the type of tissue that it is in contact with or in proximity to, and reports the information to the surgeon or enabling technology (e.g. robot) responsible for its placement. In this embodiment, one or more sensors may be located at the tip of the screw and/or one or more locations on the thread peaks. The sensing system may include any technology capable of discerning tissue type, which may include ultrasound, Fourier-transform infrared (FTIR) spectroscopy, and electrochemical sensing.

Electrochemical sensing techniques may include electrochemical impedance spectroscopy (EIS) across a range of frequencies (e.g. 1 Hz to 1 MHz), impedance measurements at a fixed frequency (e.g. 30 kHz), or electrical impedance tomography (EIT). An example application might include of a screw that with an electrode at its tip and electrodes spaced periodically along the peak of the thread that measure the type of tissue that the screw is in contact with along its length. In the case of an EIS-based system, an AC voltage is output from one or more transmitting electrodes, conducted through the surrounding tissue, and sensed at each receiving electrode. The impedance and phase shift caused by the surrounding tissue can be quantified from the signal received at each receiving electrode. This reading can be repeated across a range of AC frequencies to create an impedance profile for each type of tissue. Known tissue types can be mapped to these measured impedance profiles to create useful, real-time feedback to the surgeon or system regarding the type(s) of tissue that the screw is in contact with along its length. It should be noted that while the tissue along the length of the screw may be measured, the tissue at the tip of the screw may additionally or alternatively be measured.

One application of this technology is to provide a warning signal to prevent unsafe placement. During insertion, the screw could notify the surgeon if the tip of the screw has deviated out of a cancellous bone of the patient and penetrated a cortical wall of the pedicle, indicating an unsafe trajectory. This information could allow the surgeon to adjust the trajectory of the screw to prevent breach of the cortical wall and damage to the spinal canal. In the case of a robotic-assisted insertion of the screw, the screw could inform the system when the tip has come in contact with cerebrospinal fluid (CSF) and initiate a system lock to prevent further penetration.

Another application is to provide a verification signal to confirm intended placement. During insertion, the screw could notify the surgeon if its threads are in contact with cortical bone, indicating strong bone purchase. Alone or in combination with navigation feedback and insertion torque feedback, such a system could provide surgeons with confidence that the screws have been placed effectively.

### Intra-Op Implant Loading Feedback

Nearly all musculoskeletal implants temporarily or permanently bear loads as part of their intended function. Often, there is an ideal amount or range of loading required for the implant to provide its function for its expected life. In spine applications, loading must be balanced as best as possible between all implants to prevent stress concentrations, pullout, and breakage. To prevent subsidence of interbody cages, loads must be low enough to prevent destruction to the boney structures while sufficiently high to avoid dynamic motion and fatigue. In fracture applications, some amount of compression is required to induce osteogenesis and union along the fracture line. In total knee arthroplasty, loading should be balanced between compartments throughout the range of motion. Despite these many clinical loading applications, surgeons must rely almost entirely on subjective feel to determine if the applied loading is acceptable. There is significant opportunity for smart implants to provide loading feedback to surgeons during and after placement to address these unmet clinical needs.

In some embodiments, a smart implant is capable of providing feedback on its loading conditions. The smart implant may include one or more force sensors integrated within or attached to the body of the implant. The force sensors may include capacitive or resistance-based strain gauges, piezoelectric force sensors, multi-axis load cells, fiber Bragg grating (FBG) strain or force sensors, or force-sensitive resistors.

### Applications for Intra-Op Loading Feedback in Spine

In spine applications, loads may be needed to be balanced and optimized across individual implants as well as the construct to benefit the patient. Sensors may be located to measure loading at the implant-implant or implant-bone interfaces, including the bone-screw interface, screw-rod interface, rod-locking cap interface, interbody-endplate interface, screw-plate interface, and plate-bone interface. Sensors may also be located to enable sensing of loads within an implant's structure, including the bending and torsional stresses of a rod, bending and shear stresses of a screw, bending, torsion, and compression stresses of a plate, and bending, compression, and shear stresses of an interbody spacer.

One clinical application of this loading information includes comparison of loads between directional aspects of an interbody spacer, including distribution in the sagittal plane (e.g. anterior-posterior balance of a lateral interbody) and coronal plane (e.g. left-right balance of an anterior interbody). Such information may be particularly useful for interbodies which have more than one degree of freedom of adjustability (e.g. a lateral spacer with independent anterior and posterior expansion) to enable optimization of loading. Loading information might be presented as absolute values, relative values, heat map, static location of center of pressure, dynamic tracking of center of pressure.

Additional applications may include one or more of:
Comparison and optimization of loads between multiple interbodies within a single disc space, such as in bilateral posterior lumbar interbody fusion (PLIF);
Comparison and optimization of loads between multiple interbodies at multiple levels, such as in two-level lateral lumbar interbody fusion (LLIF).
Comparison and optimization of loads between the left and right rods at a single level (i.e. segment);
Comparison and optimization of loads between multiple segments within a single rod (e.g. left rod);
Comparison and optimization of loads between multiple segments between the left and right rods;
Comparison and optimization of global loads between left and right rods;
Comparison and optimization of loads between interbody, left rod, and right rod within a single segment; and
Comparison and optimization of loads between interbody, left rod, and right rod at multiple segments.

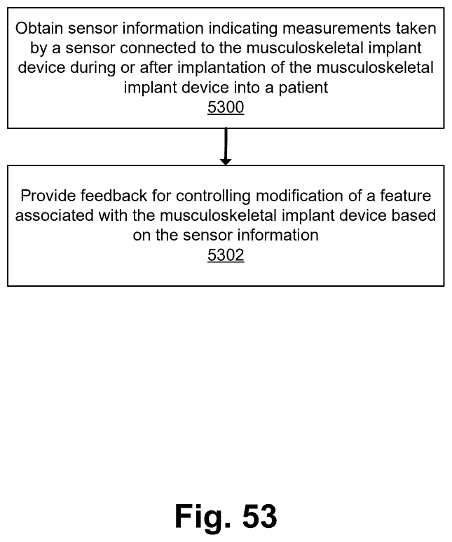

FIG. 53 illustrates a method performed by a musculoskeletal implant device, according to embodiments of the present disclosure. In some embodiments, the musculoskeletal implant device includes at least one sensor, processing circuitry, and a memory containing instructions executable by the processing circuitry, whereby the musculoskeletal implant device is operable to perform the method and embodiments disclosed herein.

In some embodiments, the musculoskeletal implant device obtains (5300) sensor information indicating measurements taken by a sensor connected to the musculoskeletal implant device during or after implantation of the musculoskeletal implant device into a patient. For example, in the above-described embodiments, the implant device may obtain sensor information indicating load values corresponding to a plurality of locations on the musculoskeletal implant device.

In some embodiments, the sensor information indicates at least one of: fusion or bone formation of a bone contacting the musculoskeletal implant device; load values corresponding to a plurality of locations on the musculoskeletal implant device; impedance measured from the musculoskeletal implant device in contract with tissue of the patient; phase in voltage measured from the musculoskeletal implant device in contract with tissue of the patient; torque on the musculoskeletal implant device; strain on the musculoskeletal implant device; biomarkers sensed by the musculoskeletal implant device; distribution of a substance in or on the musculoskeletal implant device; amount of filling of a substance in the musculoskeletal implant device; and periprosthetic joint infection.

In some embodiments, the musculoskeletal implant device provides (5302) feedback for controlling modification of a feature associated with the musculoskeletal implant device based on the sensor information. For example, the smart implant may provide the feedback to a component within the smart implant, to a controlling device external to the patient, and/or to a medical system for controlling modification of a musculoskeletal implant device.

In some embodiments, the method (e.g., steps 5300-5302) may be performed a plurality of times. For example, the obtained sensor information may be first sensor information and the feature associated with the musculoskeletal implant device is a first feature, the method of FIG. 53 may further include the musculoskeletal implant device obtaining second sensor information a time period after modifying the first feature associated with the musculoskeletal implant device, and providing feedback for controlling modification of a second feature associated with the musculoskeletal implant device based on the second sensor information. In some of embodiments, the second feature is a same or different feature as the first feature. In some of these embodiments, the time period after modifying the first feature associated with the musculoskeletal implant device is a time post-surgery of the patient (e.g., a period after the patient's muscles have had time to relax and settle).

In some embodiments, providing of feedback for controlling modification of the feature associated with the musculoskeletal implant device comprises providing feedback for controlling modification of at least one of: position of the musculoskeletal implant device implanted in the patient, orientation of the musculoskeletal implant device implanted in the patient, and expansion or contraction of a shape of the musculoskeletal implant device. In some of these or alternative embodiments, while the musculoskeletal implant device is being positioned, oriented, expanded or contracted in the patient, providing through a display device feedback indicating distribution of pressure loading across the musculoskeletal implant device based on the sensor information indicating load values corresponding to a plurality of locations on the musculoskeletal implant device.

In some embodiments, the providing of feedback for controlling modification of the feature associated with the musculoskeletal implant device comprises providing feedback for controlling modification of at least one of: electrical stimulation of a sensor electrically connected to the musculoskeletal implant device, and electrical stimulation of an electrode electrically connected to the musculoskeletal implant device.

In some embodiments, the sensor information indicates periprosthetic joint infection or bone growth, and further comprising providing the feedback to control activating, increasing, and/or decreasing vibrations generated by a vibration actuator within or connected to the musculoskeletal implant device.

In some embodiments, the sensor information indicates amount of filling of a substance in or on the musculoskeletal implant device at defined locations on the implant using impedance values measured between a plurality of spaced apart electrodes at the defined locations on the implant, and the providing of feedback for controlling includes controlling further distribution of filling of the substance in or on the musculoskeletal implant device.

In some embodiments, the providing of the feedback includes transmitting the sensor information to a controlling device that is external to the patient, and providing instructions from the controlling device to a tool configured to expand shape of the musculoskeletal implant device.

In some embodiments, the providing of the feedback includes transmitting the sensor information to a controlling device that is external to the patient; and providing instructions from the controlling device to a circuit configured to generate electrical stimulation through the musculoskeletal implant device.

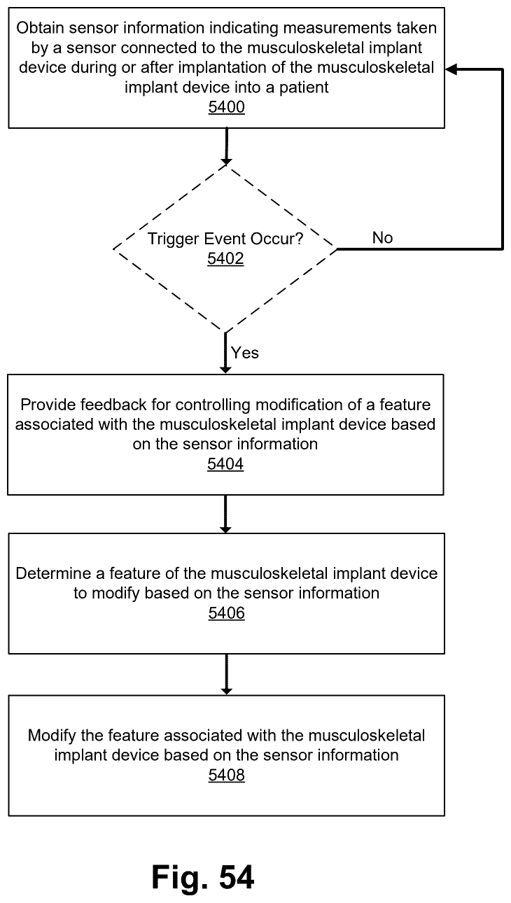

FIG. 54 illustrates another method performed by a musculoskeletal implant device in a closed-loop system, according to embodiments of the present disclosure.

In some of these embodiments, a modification of a feature of the musculoskeletal implant device is responsive to the musculoskeletal implant device determining the feature of the musculoskeletal implant device to modify based on the sensor information (e.g., closed-loop system). In this embodiment, the musculoskeletal implant device obtains (5400) sensor information indicating measurements taken by a sensor connected to the musculoskeletal implant device during or after implantation of the musculoskeletal implant device into a patient.

Optionally, the musculoskeletal implant device determines (5402) whether a trigger event occurs. A trigger event may include sensor information indicating an anomaly (e.g., joint infection, unbalanced load on the musculoskeletal implant device, sensor information above or below a threshold, threshold difference from preoperatively obtained information, etc.).

If a trigger event has not occurred, then the musculoskeletal implant device goes back to the obtaining (5400) step. If a trigger event has occurred, then the musculoskeletal implant device moves to step 5404.

In some of these embodiments, the musculoskeletal implant device provides (5404) feedback for controlling modification of a feature associated with the musculoskeletal implant device based on the sensor information. For example, in a closed-loop system, the providing of feedback would be to a component (e.g., processing circuitry) within the musculoskeletal implant device.

Then in step 5406, the musculoskeletal implant device determines a feature of the musculoskeletal implant device to modify based on the sensor information. In step 5408, the musculoskeletal implant device modifies (5408) the feature associated with the musculoskeletal implant device based on the sensor information.

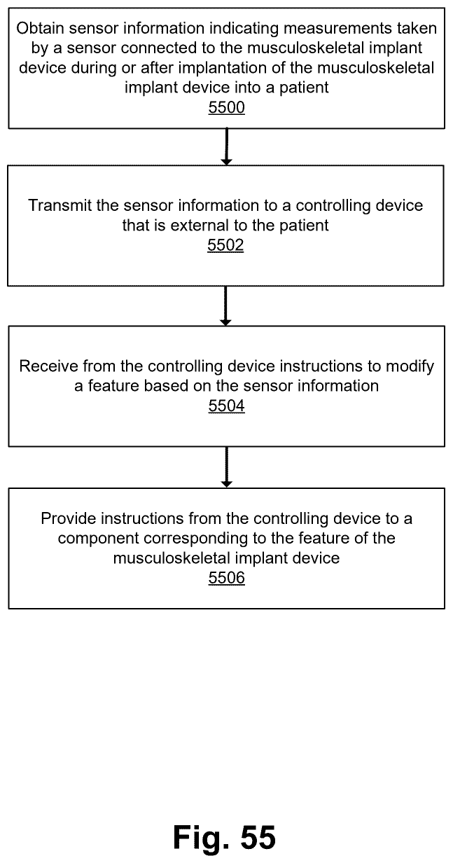

FIG. 55 illustrates yet another method performed by a musculoskeletal implant device in an open-loop system, according to embodiments of the present disclosure.

In some of these embodiments, the modification of a feature of the musculoskeletal implant device is responsive to a controlling device external to the patient determining the feature of the musculoskeletal implant device to modify based on sensor information received from musculoskeletal implant device.

In these embodiments, the musculoskeletal implant device obtains (5500) sensor information indicating measurements taken by a sensor connected to the musculoskeletal implant device during or after implantation of the musculoskeletal implant device into a patient. The musculoskeletal implant device transmits (5502) the sensor information to a controlling device (e.g., an enabling technology, a clinician device, etc.) that is external to the patient. The musculoskeletal implant device receives (5504), from the controlling device, instructions to modify a feature of the musculoskeletal implant device based on the sensor information. The musculoskeletal implant device may then provide the instructions from the controlling device to a component corresponding to the feature of the musculoskeletal implant device. For example, the component may be an actuator for expanding or contracting a shape of the musculoskeletal implant device.

In some embodiments that include open-loop applications of musculoskeletal implant devices, a medical system (also referred herein as a robotic system, enabling technology, and other systems that are discussed herein) for controlling modification of a musculoskeletal implant device includes: processing circuitry, and a memory containing instructions executable by the processing circuitry. The medical system may be operable to perform a method that includes receiving, from the musculoskeletal implant device, sensor information indicating measurements taken by a sensor connected to the musculoskeletal implant device during or after implantation of the musculoskeletal implant device into a patient. Optionally, the medical system may determine a feature of the musculoskeletal implant device to modify based on the sensor information and transmit to the musculoskeletal implant device instructions to modify a feature of the musculoskeletal implant device. Alternatively, a surgeon or clinician may determine the feature of the musculoskeletal implant device to modify based on the sensor information. Regardless, the method performed by the medical system may include transmitting modification instructions for controlling modification of a feature associated with the musculoskeletal implant device based on the sensor information.

In some of these embodiments, the sensor information indicates at least one of: fusion or bone formation of a bone contacting the musculoskeletal implant device; load values corresponding to a plurality of locations on the musculoskeletal implant device; impedance measured from the musculoskeletal implant device in contract with tissue of the patient; phase in voltage measured from the musculoskeletal implant device in contract with tissue of the patient; torque on the musculoskeletal implant device; strain on the musculoskeletal implant device; biomarkers sensed by the musculoskeletal implant device; distribution of a substance in or on the musculoskeletal implant device; amount of filling of a substance in the musculoskeletal implant device; and periprosthetic joint infection.

These musculoskeletal implant devices and medical systems are discussed in more detail both above and below.

### Sense

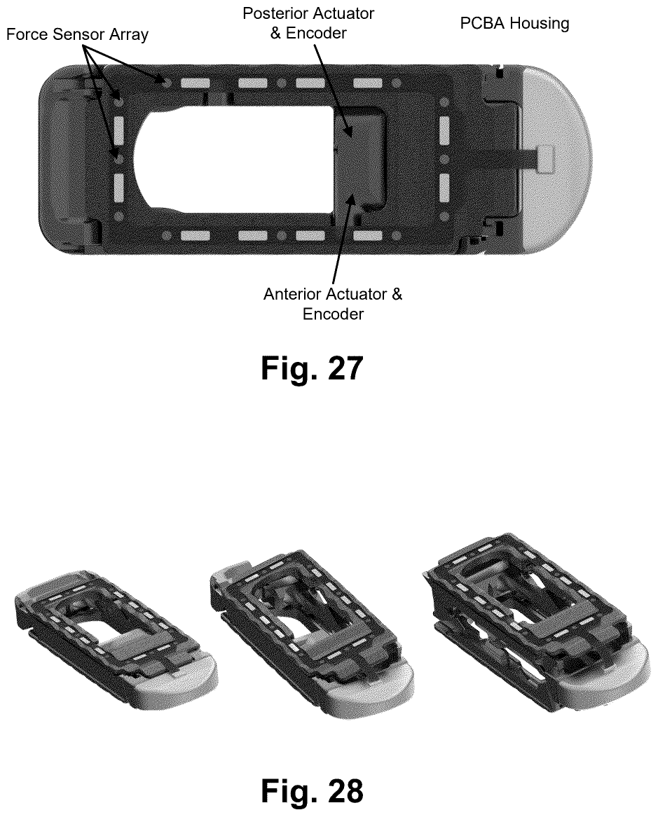

FIG. 27 illustrates a musculoskeletal implant device that includes 24 integrated force sensors enable real-time mapping of loading on the endplates, according to some embodiments of the present disclosure. While 24 integrated sensors are illustrated in FIG. 27, it should be understood that any number of force sensors may be used herein. The musculoskeletal implant device of FIG. 27 includes an expandable lateral lumbar interbody spacer with independent adjustability of anterior and posterior aspects of the implant. The anterior and posterior mechanisms may be wirelessly controlled with dedicated actuators and encoders. Superior and inferior endplates of the musculoskeletal implant device may include a force sensor array that can quantify the force distribution across the endplates. This may provide feedback to the implant, surgeon, or controlling device to identify point loading or edge loading and enables the implant, surgeon, or controlling device to determine if/what adjusts to perform, for example, anterior and/or posterior expansion to uniformly distribute the forces on the endplates. It may be desired to avoid stress concentrations to prevent implant subsidence within the adjacent vertebral endplates.

### Control

FIG. 28 illustrates independent expansion of anterior and posterior elements (IAP) of the musculoskeletal implant device with real-time feedback on implant loading, according to some embodiments of the present disclosure.

In this example, if the musculoskeletal implant device obtains information that indicates that load is not balanced of is low across the sensors (or for certain sensors) the musculoskeletal implant device may be expanded to correct the load to be with in a desired threshold or a desired range.

### Visualize

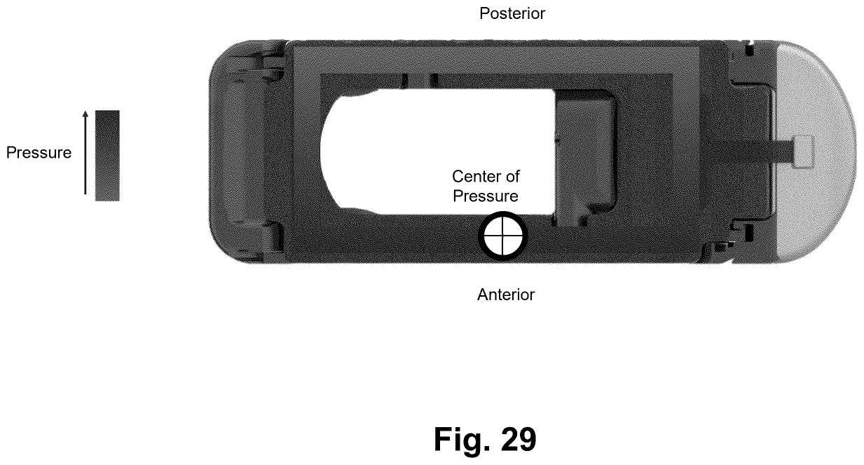

FIG. 29 illustrates a visualization of feedback regarding pressure exerted on a musculoskeletal implant device in real-time, according to some embodiments of the present disclosure.

The data may be transmitted in real-time to a software (e.g., Excelsius software) to visualize the distribution of forces and center of pressure on the musculoskeletal implant device.

### Optimize

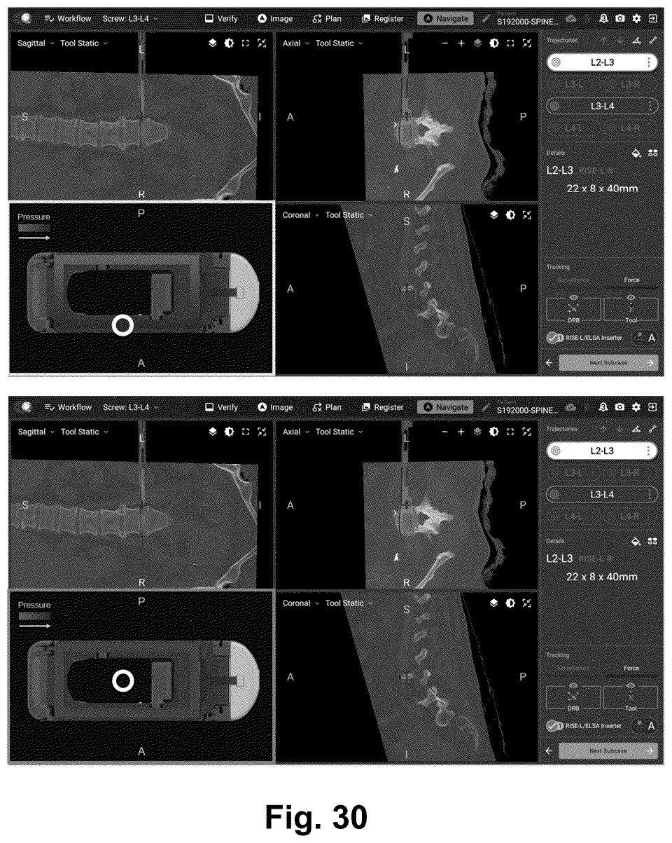

FIG. 30 illustrates another visualization of feedback regarding pressure exerted on a musculoskeletal implant device in real-time, according to some embodiments of the present disclosure. For example, FIG. 30 may be a user interface a surgeon seeing while performing a surgery.

The feedback may be used to optimize lordotic angle and endplate loading to achieve alignment goals while reducing risk of subsidence.

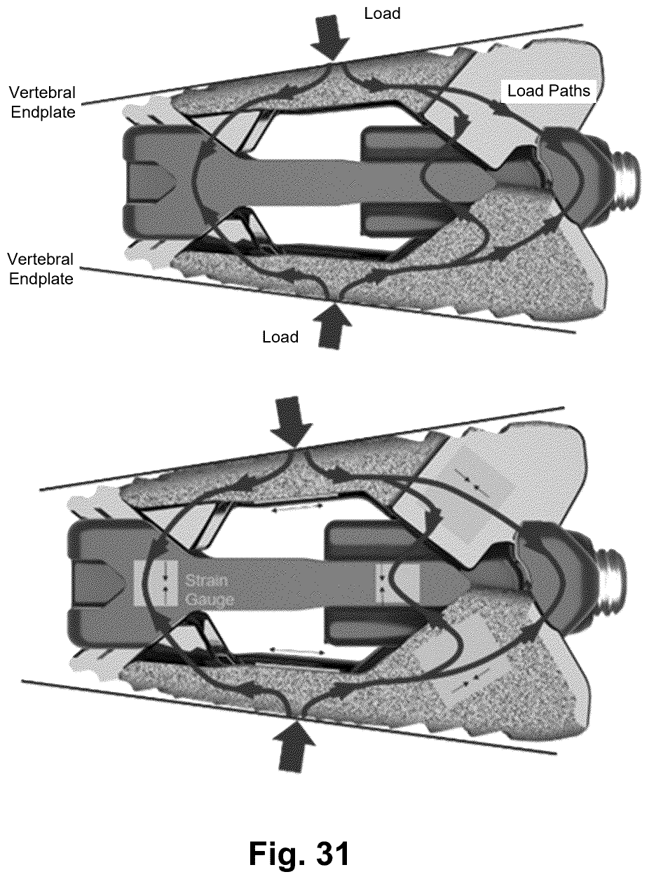

FIG. 31 illustrates load paths through a musculoskeletal implant device that includes strain gauge sensors within or on the musculoskeletal implant device, according to some embodiments of the present disclosure.

In some preferred embodiments, locations of the strain gauges on the implant correspond to sufficient available planar space to adhere strain gauge in a region that experiences the load path in the desired loading condition of measurement. For the example of the expandable TLIF spacer shown in FIG. 31, the preferred strain gauge locations are represented by boxes. The arrows in the boxes represent the direction of placement of a linear strain gauge measurement and strain direction (e.g. compression or tension). For biocompatibility and environmental protection, each strain gauge may be adhered to the implant surface with biocompatible adhesive and coated in a biocompatible epoxy or conformal coating. Electrical connections between the strain gauges and communication and control module might be facilitated by a carrier comprised of a flexible PCB made of a biocompatible material such as polyimide that is soldered to the pads of each strain gauge with biocompatible solder. The entire strain gauge and carrier assembly may be hermetically sealed in a conformal coating for biocompatibility and environmental protection.

### Applications for Intra-Op Loading Feedback in Joint Arthroplasty

In joint applications, loads are desired to be balanced between the compartments of the joint throughout the range of motion. In addition, a patient's satisfaction with the feel of their ‘new’ joint is closely correlated with this intercompartmental stiffness. If the static and dynamic loading conditions are too high, the patient may experience a ‘stiff’ feeling joint and implant components, particularly the bearing surfaces, will undergo accelerated wear. If the loading conditions are too low, the patient may experience a ‘loose’ or unstable feeling joint and implant component may experience micromotions at the implant-bone interface, which may induce an inflammatory response and degrade the surrounding bone. Force sensors may be used to measure and optimize the loading conditions on joint arthroplasty implants.

In knee arthroplasty, sensors may be located at the interfaces of the femoral component and femur, femoral component and tibial insert, tibial insert and tibial tray, tibial tray and tibia, and patellar component and femoral component. In addition, internal strain measurement may be obtained from within the femoral component, tibial insert, tibial component, or patellar component. Clinical applications include soft tissue balancing, which involves comparison of loads between the medial and lateral compartments and/or tracking the center of pressure in each compartment throughout the range of motion, as well as patella tracking, which involves tracking the center of pressure between the patella or patellar component and the femoral component throughout the range of motion. Further, these smart implant embodiments may be configured for cemented and cementless total knee, revision total knee, and unicompartmental knee replacement applications. In revision applications, additional sensor locations include the interfaces of the femoral extension stem and femur, femoral extension stem and femoral component, tibial extension stem and tibial, tibial extension stem and tibial tray, augment components and adjacent bone, and augment components and adjacent implant components.

Applications in hip arthroplasty include total hip, total revision hip, and partial hip replacement. In total hip arthroplasty, sensors may be located at the interfaces of the acetabular shell component and acetabulum, acetabular liner and acetabular shell, femoral head and acetabular liner, or femoral stem and femoral head. In revision total hip arthroplasty, additional sensor locations may include the interfaces of the femoral extension stem and femur, femoral extension stem and femoral stem, pelvic augment components and pelvis, pelvic augment components and acetabular shell component, cerclage wire and bone, cerclage wire and implant components, acetabular shell fixation screws and pelvis, and acetabular shell fixation screws and acetabular shell component. In partial hip arthroplasty, additional sensor locations may include the interfaces of the femoral head resurfacing component and acetabulum and femoral head resurfacing component and femur. Additionally, strain sensors may be integrated within each previously mentioned component to measure the internal stresses and strains.

Applications in shoulder arthroplasty include total shoulder, reverse total shoulder, and shoulder hemiarthroplasty. In total shoulder arthroplasty, sensors may be located at the interfaces of the humeral head component and humeral stem component, humeral stem component and humerus, humeral head component and glenoid fossa component, glenoid fossa component and glenoid. In reverse total shoulder arthroplasty, sensors may be located at the interfaces of the humerus and humeral stem component, humeral stem component and humeral cup component, humeral cup component and glenoid sphere component, and glenoid sphere component and glenoid. In shoulder hemiarthroplasty, sensors may be located at the interfaces of the humerus and humeral head resurfacing component and humeral head resurfacing component and glenoid fossa. Additionally, strain sensors may be integrated within each previously mentioned component to measure the internal stresses and strains.

In total elbow arthroplasty, sensors may be located at the interfaces of the humerus and humeral component, humeral component and humeral bearing liner component, humeral bearing liner component and hinge pin component, hinge pin component and ulnar bearing liner component, ulnar bearing liner component and ulnar component, and ulnar component and ulna. Additionally, strain sensors may be integrated within each previously mentioned component to measure the internal stresses and strains.

In total ankle arthroplasty, sensors may be located at the interfaces of the talus and talar component, talar component and bearing component, bearing component and tibial component, and tibial component and tibial. Additionally, strain sensors may be integrated within each previously mentioned component to measure the internal stresses and strains.

In total wrist arthroplasty, sensor may be located at the interfaces of the radius and radial component, radial component and carpal ball component, carpal ball component and carpal plate component, carpal plate component and metacarpal screws/stems, and metacarpal screws/stems and metacarpal bones. Additionally, strain sensors may be integrated within each previously mentioned component to measure the internal stresses and strains.

In the previously described arthroplasty applications, the loading feedback may be used intraoperatively to assess fixation between the implant and mating bone or cement, load balance through range of motion, center of pressure tracking throughout range of motion, and stability through range of motion.

Further, assessment of the stiffness, range of motion, and loading conditions of the native joint may be conducted preoperatively or in the initial stage of the procedure and compared to the data obtained from the smart implants to determine if the treated joint matches the clinical parameters of the native knee and treatment plan. In some embodiments, the trigger event may occur when the obtained sensor information is a threshold difference from this preoperatively obtained information.

Preoperative data may be collected through the following means:
Wearable devices, including:
   Sensors mounted to the skin around the target joint,
   Sensors mounted to a brace or piece of clothing worn around the joint, and/or
   Sensor mounted within an insole placed in the patient's shoe;
Devices that monitor a patient's motion in the patient's home or clinic such as an optical camera system and pressure sensing mat;
A smart pressure sensing glove that a clinician wears which can measure the applied force while the manipulating the extremity/joint; and/or
An enabling technology, such as a robotic system or smart bed, which may be used to apply controlled motions to the joint and measure the force throughout the range of motion.
   In the case of knee or hip arthroplasty, the patient's foot or leg may be rigidly fixated in a boot or brace which is coupled to the robot or smart table end effector.

### Applications for Intra-Op Loading Feedback in Trauma

In trauma applications, loads must be balanced and optimized across individual implant components and construct to ensure there aren't excessive stress concentrations which could cause fracture or fatigue of the implants or bone. Additionally, loads are of interest to ensure that ample compression is applied at the bone fracture lines to induce osteogenesis. If compression is too low, high micromotions may cause osteolysis and non-union. If the compression is too high, micromotions may be nonexistent which may slow the bone formation process. Therefore, it would be helpful for surgeons to be able to measure various loads intraoperatively to ensure the construct is optimized for long-term durability of the implant components and union at the fracture site.

Sensors may be located in such a way to measure loading at the implant-implant or implant-bone interfaces, including the bone-screw interface, screw-plate interface, bone-plate interface, bone-nail interface, nail-screw interface, wire-plate interface, and wire-bone interface. Sensors may also be located to enable sensing of loads within an implant's structure, including the compression, tension, shear, bending and torsional stresses of a screw, plate, nail, or wire.

### Intra-Op Graft Delivery Feedback

In spine surgery, graft is often backfilled within the graft window of an expandable spacer after the spacer is placed and expanded. The implant graft window is typically packed with graft material prior to insertion, however, the graft window volume increases when the device is expanded, which opens additional available space for graft material to be placed via backfilling. Historically, surgeons have attempted to estimate the amount of graft material delivered to window by measuring the inserted volume and comparing it to an estimated available volume from the implant geometry. Realistically, surgeons often place as much graft as possible until they are met with resistance that prevents them from placing additional material. In both scenarios, the surgeon has no real understanding of the density or location of graft within the implant. In the case where the surgeon is met with resistance that prevents further delivery, analysis of the distribution (e.g., density and location) of delivered graft may reveal a gap within the volume, which may be targeted by tamping or probing the graft material inside the implant with miniature instruments. There is an opportunity for smart implants to provide such feedback intraoperatively to enable the surgeon to know that sufficient graft has been placed throughout the implant, which is critical for formation of a boney bridge and fusion between vertebral bodies.

In some embodiments, the smart musculoskeletal implant is capable of assessing graft placement. In some embodiments, the musculoskeletal implant includes an interbody device with force or strain sensors attached to or integrated within its structure. The force sensors may include capacitive or resistance-based strain gauges, piezoelectric force sensors, multi-axis load cells, fiber Bragg grating (FBG) strain or force sensors, or force-sensitive resistors. Regardless of the implementation, the implant may report the measured force to the surgeon via absolute values, relative values, a heatmap, or combination of these.

It should be noted that, herein, when information is reported, transmitted, or otherwise provided to the surgeon, it may additionally or alternatively be provided to the robotic system for robotic assisted or fully autonomous surgeries.

The surgeon or system could save a baseline force reading prior to the backfilling step. During or after the graft window is backfilled, additional measurements could be captured to note the change in distribution (e.g., force). As the graft window is backfilled, the graft material may take some of the load off of the interbody device and the measured load may be less than the baseline value. This difference in force can then be compared to predetermined thresholds to determine whether or not the graft window is sufficiently packed.

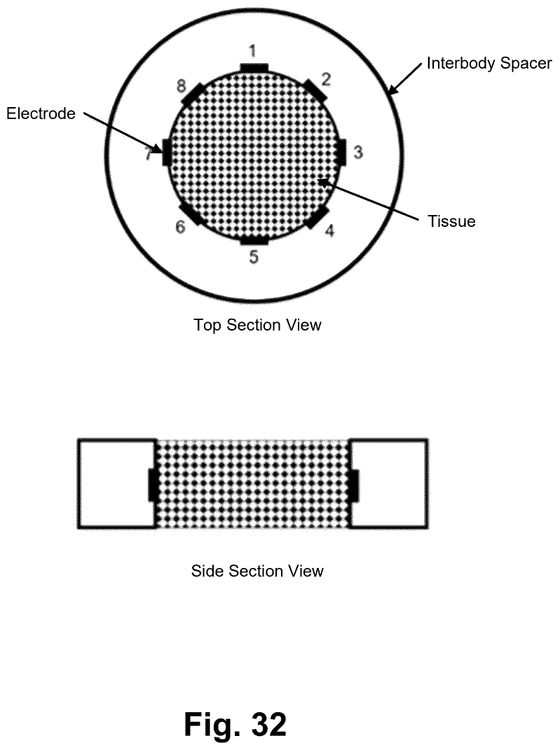

Another embodiment includes of an interbody device with sensors attached to or integrated within the interior surface of the graft window. The sensing system includes technology capable of discerning tissue type (e.g., ultrasound, Fourier-transform infrared (FTIR) spectroscopy, and electrochemical sensing). Electrochemical sensing techniques may include electrochemical impedance spectroscopy (EIS) across a range of frequencies (e.g. 1 Hz to 1 MHz), impedance measurements at a fixed frequency (e.g. 30 kHz), or electrical impedance tomography (EIT). An example embodiment includes an interbody spacer with electrodes spaced periodically along the surface of graft window in a single plane, as shown in FIG. 32.

FIG. 32 illustrates a top sectional view and a side view of an interbody spacer with electrodes spaced periodically along a surface of a graft window in a single plane, according to some embodiment of the present disclosure.

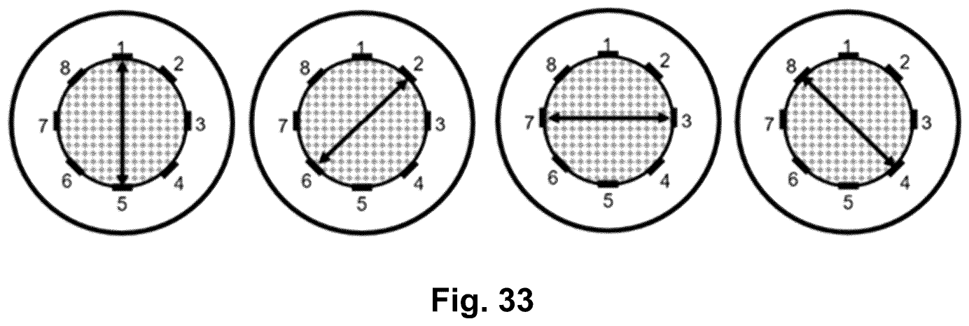

FIG. 33 illustrates a top sectional view of an interbody spacer that sequentially uses different electrodes to measure the filling of graft material, according to some embodiments of the present disclosure.

In the case of a fixed frequency impedance measurement system, an AC voltage at a known frequency (e.g. 10 kHz) is output from a transmitting electrode, conducted through the tissue within the graft window, and sensed at the receiving electrode. The impedance and phase shift caused by the tissue can then be quantified from the signal at the receiving electrode. This reading can be correlated to a specific type of tissue (e.g. bone or fluid). Further, the measured impedance and phase shift can also be correlated to bone mineral density (BMD). The BMD can then be used to estimate how filled the graft window is with graft material (e.g. 50% filled). Additionally, other measurements across the measurement plan can be determined by alternating which pairs of electrodes are used for the measurement. In the example shown in FIG. 33, the system measures the impedance between electrodes 1 and 5, followed by 2-6, 3-7, and 4-8. Collectively, these measurements can be used to determine and then inform the surgeon that the graft window is, for example, ˜100% filled.

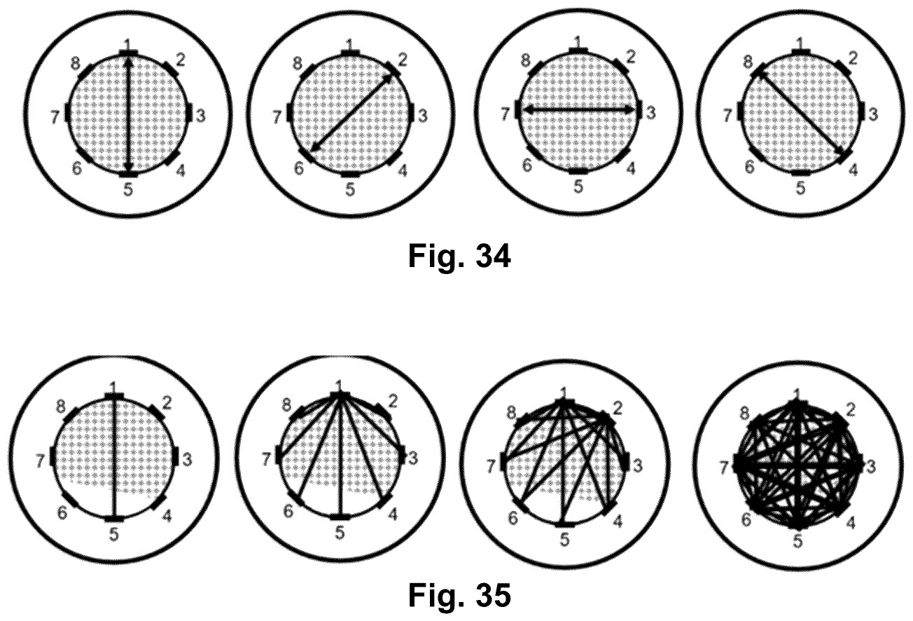

FIG. 34 illustrates a top sectional view of another interbody spacer that sequentially uses different electrodes to measure the filling of graft material, according to some embodiments of the present disclosure.

In the example shown in FIG. 34, the graft window contains a gap near electrodes 4, 5, and 6. By repeating the previously described measurement, the system could determine and then inform the surgeon that the graft window is ˜80% filled.

FIG. 35 illustrates a top sectional view of yet another interbody spacer that uses different electrodes to measure the filling of graft material and determine the location of a gap in the graft material, according to some embodiments of the present disclosure.

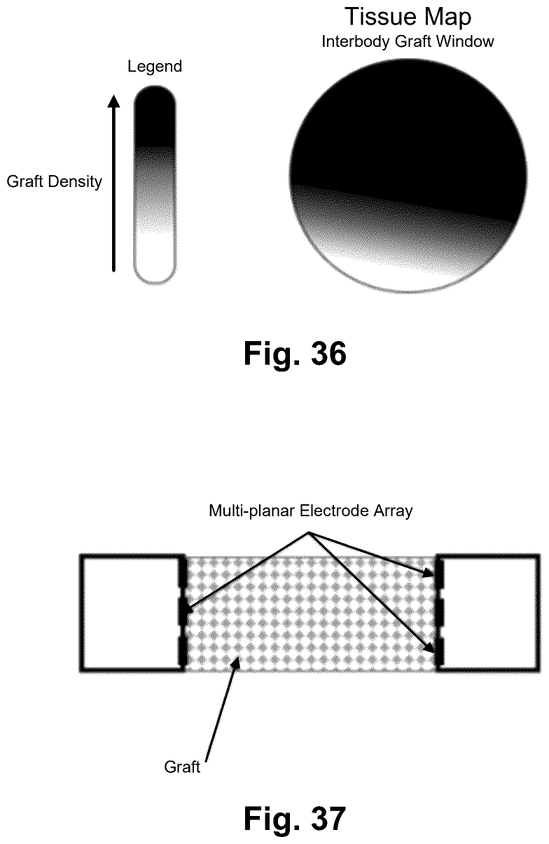

In addition to the percent filled, the surgeon may also wish to know where the graft/gap is located in order to try to adjust the placed graft within the window to enable more to be placed. Electrical impedance tomography (EIT) techniques can be employed to reconstruct an image or heatmap from impedance measurements captured between every combination of electrodes, as shown in FIG. 36.

FIG. 36 illustrates an example of a two-dimensional map of tissue within a graft window, according to embodiments of the present disclosure.

From the EIT data, a two-dimensional map of the tissue within the graft window can be constructed and presented to surgeon. The surgeon can then use this information to adjust the position of graft within the window to enable additional backfilling.

The previously described embodiments over a 1D and 2D implementation, however, surgeons may also be interested in the 3D volumetric information. The 2D EIT techniques can be applied for 3D mapping of the graft window by the inclusion of a multiplanar electrode array.

FIG. 37 illustrates a side section view of a multiplanar electrode array, according to some embodiments of the present disclosure.

From the EIT data, a three-dimensional map of the tissue within the graft window can be constructed and presented to surgeon. The surgeon can then use this information to adjust the position of graft within the window to enable additional backfilling.

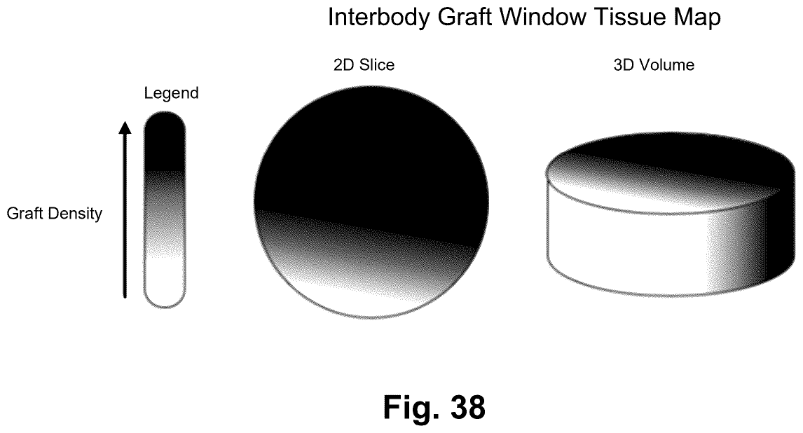

FIG. 38 illustrates an example of a two-dimensional map and a three-dimensional map of tissue within a graft window, according to some embodiments of the present disclosure.

In some embodiments, this process may also be applied to determine the amount and location of graft placed around an interbody spacer within the disk space. These embodiment may include one or more planes of electrodes integrated periodically along the exterior surface(s) of the interbody spacer.

### Post-Placement Adjustment

In some clinical scenarios, a surgeon may want to alter the position, state, or loading of implant components without requiring the implant insertion or adjustment instruments to be attached to the implant.

One embodiment of post-placement adjustment is passively enabled by actuation of a locking mechanism that locks and unlocks a preloaded expansion mechanism. An example embodiment includes a smart interbody spacer that has a spring-loaded expansion mechanism, which provides the surgeon the ability to adjust the height of the interbody after the inserter and access port are removed. After interbody placement, the surgeon may choose to compress the posterior screw heads to achieve additional lordotic correction. With a traditional implant, the implant could become unevenly or insufficiently loaded due to changes in loading conditions. The spring-loaded expansion mechanism of the proposed implant could allow the implant to self-adjust to fit the new disc height and/or lordotic angle. When the implant and anatomy are in the desired position, the surgeon could then activate a locking mechanism within the smart interbody to rigidly fix the new position. This locking mechanism could be actuated via a small nitinol rod that is powered by a wired tether that passes through the now collapse transforaminal access corridor to an external control device.

In other embodiments of this passive configuration, the powered locking system could be driven by an electric motor, electric motor with external stator (e.g. MAGEC), shape memory alloy (SME) linear actuator, SME torsional actuator, magnetostricitve actuator (e.g. Nitinail), hydraulic pump, pneumatic pump, solenoid, or piezoelectric walk drive. Additionally, the force/torque output of the actuator could be amplified by a planetary gearbox, worm gearbox or other transmission element. Finally, the preloaded expansion mechanism might include a spring-loaded expansion mechanism or compliant mechanism.

Smart implant systems for post-placement adjustment may also be actively enabled by direct actuation of an expansion mechanism. An example embodiment includes a smart interbody spacer that has a motor-driven, geared expansion mechanism that provides the surgeon the ability to adjust the height of the interbody after the inserter and access port are removed. The surgeon may choose to compress the posterior screw heads to achieve additional lordotic correction. When this compression occurs, the loading conditions on the implant change and the implant could be unevenly or insufficiently loaded. The surgeon could adjust the implant to fit the new disc height by activating the drive motor via a wired tether that passes through the now collapsed transforaminal access corridor to an external control device, such as a robotic navigation system. The implant could also provide feedback to the surgeon regarding its kinematic state, such as the expansion height and lordotic angle.

In some embodiments of the active configuration, the powered drive system is actuated by an electric motor, electric motor with external stator (e.g. MAGEC), shape memory alloy (SME) linear actuator, SME torsional actuator, magnetostricitve actuator (e.g. Nitinail), hydraulic pump, pneumatic pump, solenoid, or piezoelectric walk drive. Additionally, the force/torque output of the actuator could be amplified by a planetary gearbox, worm gearbox or other transmission element.

### Balance

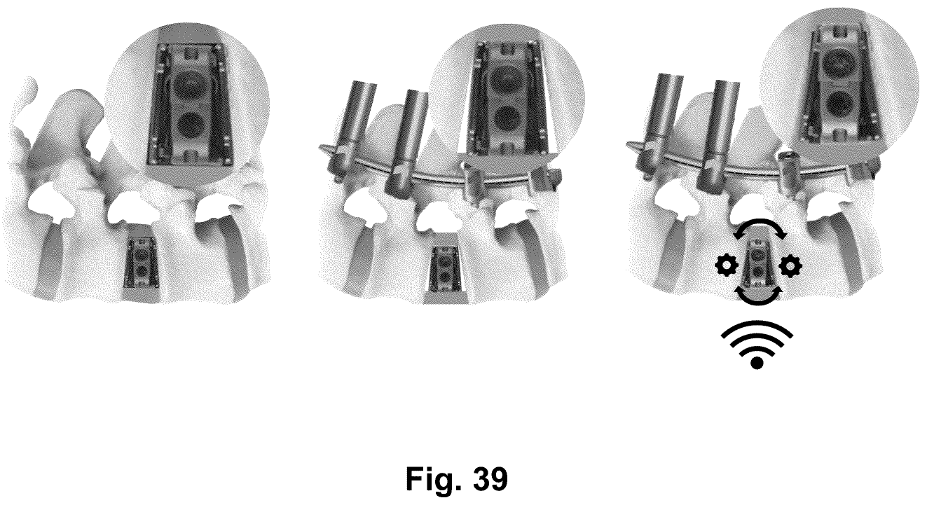

FIG. 39 illustrates an application of a smart implant for post-placement adjustment, according to some embodiments of the present disclosure. The smart implant may be used for balance construct alignment and load adjustment to account for changes during posterior fixation.

Spine applications of the smart implant for post-placement adjustment include optimization of load sharing across an entire construct, optimization of segmental sagittal loading and alignment, optimization of segmental coronal loading and alignment, optimization of pressure distribution between an interbody and adjacent vertebral endplate, and optimization of loading at the bone-screw interfaces. The described clinical objectives could also occur at multiple stages of the procedure including after the first interbody is placed, after all interbodies are placed, and/or after all interbodies and rods are placed. Adjustable implants may include interbody spacers and posterior fixation implants. A smart adjustable interbody spacer may enable post-placement adjustment of the overall height, footprint, independent heights of anterior and posterior aspects (sagittal angle), and independent heights of left and right elements (coronal angle). Smart adjustable posterior fixation implants may enable post-placement adjustment of the distance between screw heads (i.e. length of rod), angle between the screw and rod, and length of screw.

Trauma applications of a smart implant for post-placement adjustment include optimization of load sharing across the implant construct, optimization of compression of the fracture gap, and optimization of anatomic alignment of fracture components. Adjustable smart implants for trauma applications may include plates, screws, and nails. A smart adjustable plate may enable post-placement adjustment of the length of the plate, angle between the plate and a screw, angle between proximal and distal aspects of the plate. A smart adjustable screw may enable post-placement adjustment of the screw length to optimize compression and/or prominence. A smart adjustable nail may enable post-placement adjustment of the nail length, angle between a screw and the nail, angle between the proximal and distal elements of the nail, and diameter/fit of the nail within the canal.

Joints applications of a smart implant for post-placement adjustment include knee and hip. Knee applications include optimization of load sharing between medial and lateral compartments, optimization of overall soft tissue balance through range of motion, optimization of center of pressure through range of motion, and optimization of patella tracking. Hip applications include optimization of distance between center of rotation and femur axis, optimization of fit of stem within canal, optimization of leg length, and optimization of acetabular shell opening angle. Smart implants for knee include the tibial insert, tibial tray and femoral component. Mechanisms within one or more of these components could enable adjustment of varus-valgus angle, medial compartment height, lateral compartment height, flexion gap, and extension gap. Smart adjustable implants for hip include the femoral stem and cup/shell. Mechanisms within one or both of these components could enable adjustment of leg length, offset length, offset angle, fit between stem and femur, and cup angle.

Postoperative Applications—Smart Implants that Enhance Surgical Outcomes

### Postoperative Adjustment

Smart implants for postoperative adjustment enable wireless, noninvasive adjustment of the implant components after surgery. One embodiment is passively enabled by actuation of a locking mechanism that locks and unlocks a preloaded expansion mechanism. An example includes a smart interbody spacer that has a spring-loaded expansion mechanism that provides the surgeon the ability to adjust the height of the interbody after surgery to restore loss of disc height due to subsidence or changes in loading conditions. When the patient is transferred from the prone position on the OR table to a supine position in bed, the loading conditions on the implant change and the implant could be unevenly or insufficiently loaded. The spring-loaded expansion mechanism of the implant could allow the implant to self-adjust to fit the new disc height. The surgeon could unlock the expansion mechanism via a wireless actuator or wireless power system. The surgeon could then control the amount of expansion by placing the patient in a special chair or bed which permits controlled articulation of lordosis. When the implant and anatomy are in the desired position, the surgeon could then activate a locking mechanism within the smart interbody to rigidly fix the new position. This locking mechanism could, for example, be actuated via a small nitinol rod that is powered by wireless external control device.

In other embodiments of this passive configuration, the powered drive system could be actuated by an electric motor, electric motor with external stator (e.g. MAGEC), shape memory alloy (SME) linear actuator, SME torsional actuator, magnetostricitve actuator (e.g. Nitinail), hydraulic pump, pneumatic pump, solenoid, or piezoelectric walk drive. Additionally, the force/torque output of the actuator could be amplified by a planetary gearbox, worm gearbox or other transmission element. Finally, the preloaded expansion mechanism might include a spring-loaded expansion mechanism or compliant mechanism.

In another embodiment, the smart implant system may be actively enabled by direct actuation of an expansion mechanism. An example includes a smart interbody spacer that has a motor-driven, geared expansion mechanism that provides the surgeon the ability to adjust the height of the interbody after surgery to restore loss of disc height due to subsidence or changes in loading conditions. When patient is transferred from the prone position on the OR table to a supine position in bed, the loading conditions on the implant change and the implant could be unevenly or insufficiently loaded. The surgeon could adjust the implant fit within the new disc height/angle by activating the drive motor via a wireless external control device, such as a smart wireless power mat. These adjustments may be responsive to receiving and viewing sensor information that is obtained from the smart implant. The implant could also provide sensor information (i.e., feedback) to the surgeon regarding its kinematic state, such as the expansion height and lordotic angle.

In other embodiments of this active configuration, the powered drive system could be actuated by an electric motor, electric motor with external stator (e.g. MAGEC), shape memory alloy (SME) linear actuator, SME torsional actuator, magnetostricitve actuator (e.g. Nitinail), hydraulic pump, pneumatic pump, solenoid, or piezoelectric walk drive. Additionally, the force/torque output of the actuator could be amplified by a planetary gearbox, worm gearbox, or other transmission element.

### Adapt

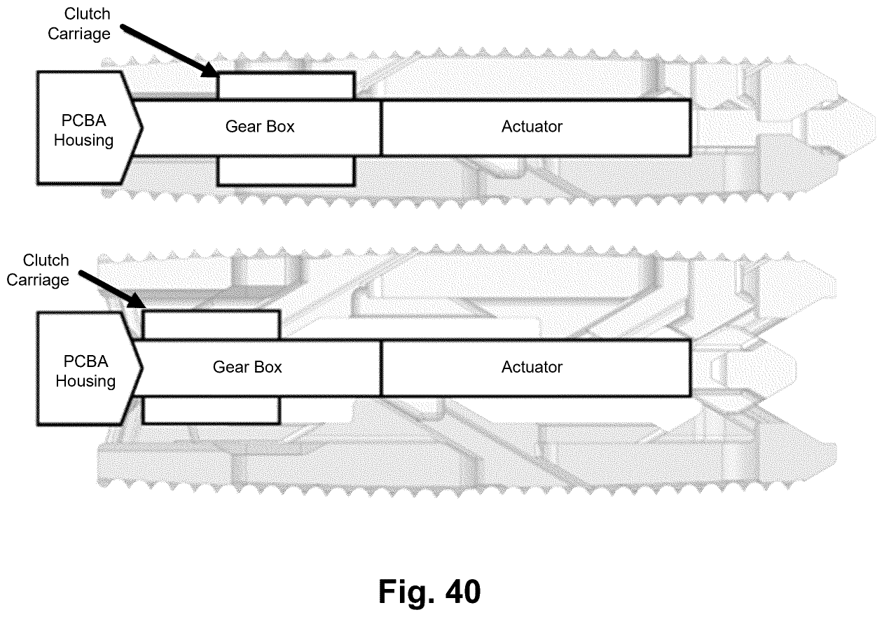

FIG. 40 illustrates yet another smart implant that is able to expand to account for post-operative settling and relaxation of soft tissue, according to some embodiments of the present disclosure.

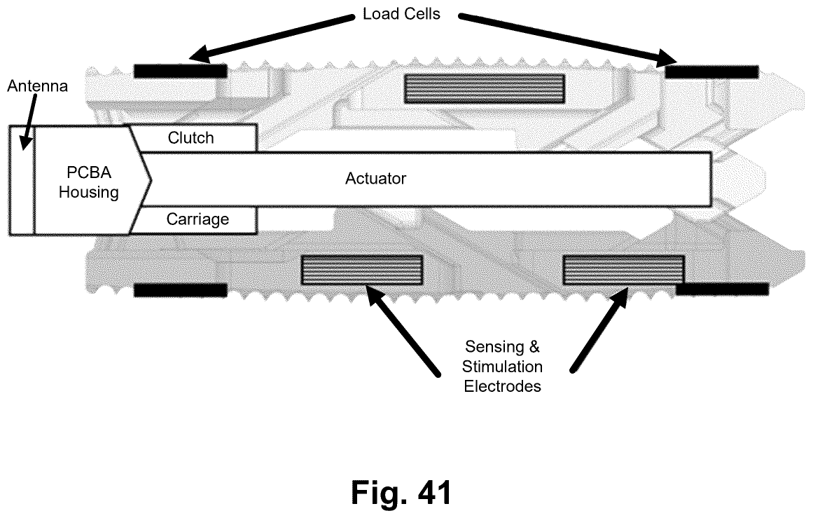

FIG. 41 illustrates yet another smart implant that is also able to expand to account for post-operative settling and relaxation of soft tissue and includes sensing and stimulation electrodes, according to some embodiments of the present disclosure.

Spine applications of a smart implant for postoperative adjustment include optimization of load sharing across an entire construct, optimization of segmental sagittal loading and alignment, optimization of segmental coronal loading and alignment, and optimization of pressure distribution between an interbody and adjacent vertebral endplate, and optimization of loading at the bone-screw interfaces. The described clinical objectives could also occur at multiple stages in the recovery period, including immediately after the patient is flipped to the supine position in the OR, after the patient stands for the first time, or 2 weeks after surgery in the first follow-up visit. Adjustable implants may include interbody spacers and posterior fixation implants. A smart adjustable interbody spacer may enable post-placement adjustment of the overall height, footprint, independent heights of anterior and posterior aspects (sagittal angle), and independent heights of left and right elements (coronal angle). Smart adjustable posterior fixation implants may enable post-placement adjustment of the distance between screw heads (i.e. length of rod), angle between the screw and rod, and length of screw.

Trauma applications of a smart implant for postoperative adjustment include optimization of load sharing across the implant construct, optimization of compression of the fracture gap, and optimization of anatomic alignment of fracture components. Adjustable smart trauma implants may include plates, screws, and nails. A smart adjustable plate may enable post-placement adjustment of the length of the plate, angle between the plate and a screw, angle between proximal and distal aspects of the plate. A smart adjustable screw may enable post-placement adjustment of the screw length to optimize compression and/or prominence. A smart adjustable nail may enable post-placement adjustment of the nail length, angle between a screw and the nail, angle between the proximal and distal elements of the nail, and diameter/fit of the nail within the canal.

Joints applications of a smart implant for postoperative adjustment include knee and hip. Knee applications include optimization of load sharing between medial and lateral compartments, optimization of overall soft tissue balance through range of motion, optimization of center of pressure through range of motion, and optimization of patella tracking. Hip applications include optimization of distance between center of rotation and femur axis, optimization of fit of stem within canal, optimization of leg length, and optimization of acetabular shell opening angle. Smart adjustable implants for knee include the tibial insert, tibial tray and femoral component. Mechanisms within one or more of these components could enable adjustment of varus-valgus angle, medial compartment height, lateral compartment height, flexion gap, and extension gap. Smart adjustable implants for hip include the femoral stem and cup/shell. Mechanisms within one or both of these components could enable adjustment of leg length, offset length, offset angle, fit between stem and femur, and cup angle.

Postoperative Applications—Smart Implants that Detect, Prevent and Treat Complications

### Acceleration of Bone Growth & Prevention of Bone Growth Issues

### Pseudarthrosis Following Lumbar Interbody Fusion

In lumbar interbody fusion, a rigid spacer is placed within the disc space to restore the disc height, correct global alignment, stabilize the construct, and assist in the bone modeling. After the interbody device is placed, the vertebral body endplates are loaded in a higher than normal condition that causes the formation of new and denser bone in accordance with Wolff's Law. Solid fusion is expected to occur between three and six months after surgery, when new bone originating from the cranial and caudal endplates connect to form a continuous bridge. To increase the probability of fusion, surgeons may use a MIS TLIF and developed implant innovations such as expandable devices and 3D printed porous titanium spacers. Despite these advances, pseudarthrosis may remain a concern in spinal surgery.

Pseudarthrosis is commonly identified as the primary cause for revision following lumbar interbody fusion. Pseudarthrosis, also referred to as non-union or non-fusion, is typically defined as a failure of solid fusion one year after surgery. There are a number of surgical, mechanical, biological, and patient factors that contribute to non-union. Prominent factors include poor surgical technique, metabolic abnormalities, excessive motion of the construct, trauma, infection, and smoking. Smoking is one of the most significant and controllable contributing factors to spine fusion. Non-union rates for smokers have been reported as high as 40% compared to 8% for non-smokers. Smoking has been correlated with lower-than-normal perfusion and oxygenation which inhibits bone growth, and nicotine specifically has been shown to inhibit revascularization of cancellous bone graft. Delayed union and non-union can be treated non-surgically through non-invasive electrical bone growth stimulation, which has been shown to increase osteogenic activity through electrical signaling. Operative treatment is often only considered if the pseudarthrosis is symptomatic or causes a mechanical instability or failure of the implant hardware. Full revision of the construct is often reserved as a last resort due to relatively poor results, with rates of recurrent pseudarthrosis of up to 50% and clinical failure in as many as 70% of cases.

Smart implants could play a significant role in assisting clinicians by continuously and quantitatively monitoring the development of fusion, remotely detecting delayed healing and complications, and diagnosing subsequent pathologies. For example, a smart interbody spacer could be used to monitor the formation of bone and degree of fusion through onboard bioimpedance sensors and load cells. In addition, the force data could be used to quantify the loading distribution on the implant and endplates. By integrating kinematic data from accelerometers or displacement sensors, the implant could also measure the construct mobility and stiffness in situ. Further, detection algorithms could be incorporated to alert the care team of delayed healing trends, excessive mobility, and hardware failure and autonomously diagnose the pathologies. With this information at their fingertips, surgeons could enhance their assessment of healing and timing of treatment decisions. The population data could also be useful for quantifying the effectiveness of various treatment strategies, surgical plans, and implant designs.

In addition to providing unparalleled information about the incidence of pseudarthrosis, smart implants could also serve a therapeutic role in treating delayed fusion or non-union through direct current electrical stimulation. Direct current electrical stimulation (DCES) is an implantable adjunct treatment option for increasing the probably of spine fusion and long bone unions. DCES may be advantageous over non-invasive electrical stimulation due to compliance-independent treatment directly at the site of fusion. In a study of 118 patients who had a DCES device implanted following multilevel pseudarthrosis, 92% of patients achieved successful fusion. In a study of high-risk patients, a clinical success rate of 91% was reported for a group of patients who received implantable bone growth stimulators compared to 79% in the non-stimulation group. Widespread adoption of DCES as an adjunct for lumbar interbody fusion has been minimal due to the lack of sufficient clinical evidence. Usage and rigorous study are likely limited due to the existence of only one product offering in this category, which is designed for use in posterolateral fusion. Usage will continue to be limited until these devices are configured in a manner that is effective and easy to use in interbody fusion.

Smart interbody devices could provide the demonstrated therapeutic benefits of DCES in an accepted form factor and workflow. The addition of DCES technology into static and expandable interbody spacers, including the next generation 3D printed expandables, could further enhance the clinical effectiveness of these innovative implants. The clinical benefits are particularly significant for patients with high-risk factors, including multi-level constructs, revision surgery, obesity, and a history of smoking. The smart interbody implants could be offered in an open-loop configuration where a constant dose is delivered for approximately six months following surgery, which is similar to the protocol with existing devices. With the addition of the previously described sensors, the implant could also monitor the effects of stimulation on the progression of fusion and enhance the dose if needed. This feedback loop could also be controlled autonomously by the implant, allowing the implant to adapt the therapy to the needs of each individual patient.

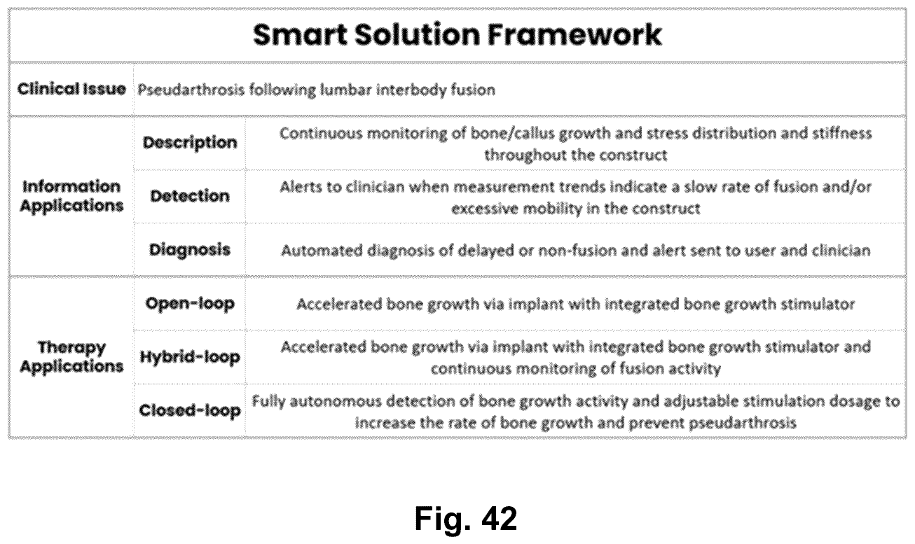

FIG. 42 illustrates a smart solution framework chart for pseudarthrosis following lumbar interbody fusion, according to some embodiments of the present disclosure.

### Accelerate

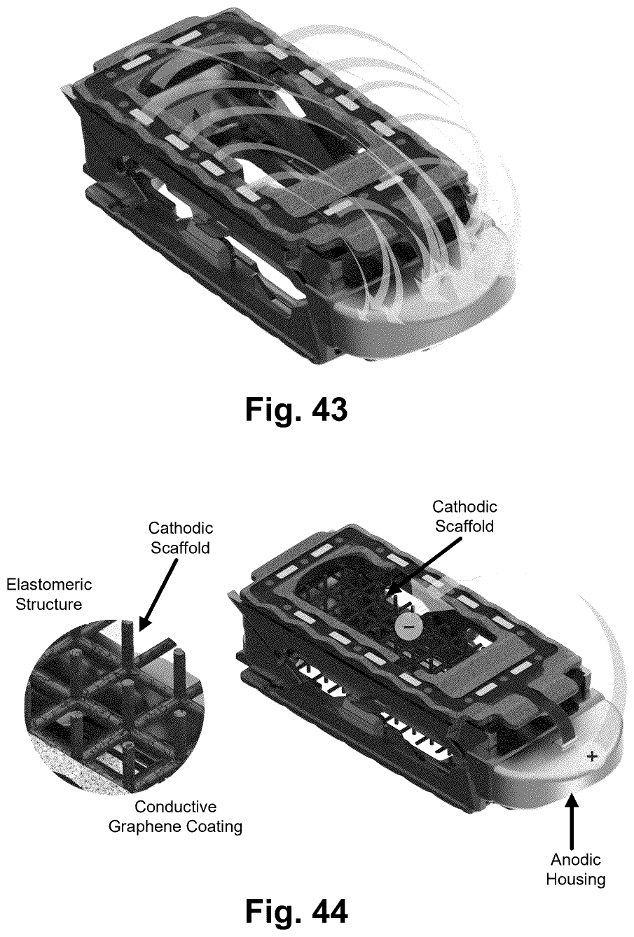

FIGS. 43-44 illustrate a smart implant with a conductive graft scaffold, according to some embodiments of the present disclosure. The smart implant of FIGS. 43-44 may accelerate bone formation across a disc space with the conductive graft scaffold. The conductive graft scaffold may act as a three-dimensional (3D) electrode to deliver and distribute electrical bone growth stimulation through the implant graft window. The scaffold may be advantageous over electrodes integrated into the interbody implant surfaces because the electric field generated by the conductive graft scaffold may be more uniform across and targeted to the graft material. An elastomeric structure may provide the ability to collapse to a compact shape during insertion and expand along with the interbody during an implant expansion operation. Generally, elastomeric materials are non-conductive, so the graphene coating can render the elastomeric structure as conductive.

A portion of the smart implant may include an anodic housing that is configured to create an anode charge and the graft scaffold may be configured to create a cathode charge. At a certain voltage and/or frequency, the electric field formed between the anodic housing and the graft scaffold may accelerate bone formation across the disc space.

### Monitor

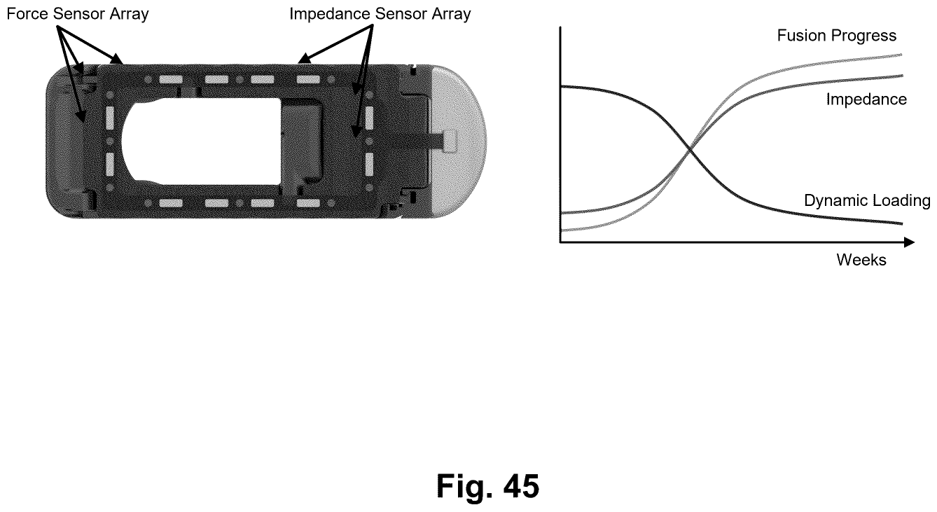

FIG. 45 illustrates fusion progress, impedance, and dynamic loading sensed over time by a smart implant, according to some embodiments of the present disclosure. The smart implant may monitor fusion progress by sensing (through the force sensor array and impedance sensor array) changes in implant loading and tissue impedance over time.

### Additional Applications and Embodiments for Bone Growth

The following smart implant embodiments are intended to aid in the acceleration of bone growth activity at the site where fusion is desired. Applications of these smart implants include nearly all musculoskeletal procedures and implant types, including fracture union in trauma, vertebral body fusion in spine, and bone in-growth and on-growth in spine and joint arthroplasty. The below smart implants include therapeutic and sensing embodiments.

### Therapy

One set of embodiments includes smart implants that mechanically-induce or enhance osteogenic activity via ultrasonic vibrations. Active vibration of an implant may be accomplished through an internal actuator mechanism or an actuator mechanism connected to the smart implant, which may include a piezo transducer, eccentric rotating mass (ERM), linear resonant actuator (LRA), or magnetostrictive transducer. Micromotions at the targeted site of bone growth (e.g. fracture line) may be also be induced passively through a concept called reverse dynamization. One embodiment of a smart implant capable of reverse dynamization includes a compliant mechanism at or near the targeted site of bone growth that can be locked non-invasively in the postoperative period. An example of the compliant mechanism includes a planar parallel compliant mechanism with a bistable compliant mechanism switch. An example of this embodiment is a fracture plate that contains a compliant mechanism within its structure that is in line with the fracture line. In the immediate post-op period, the compliant mechanism is unlocked which permits micromotions between the fracture interfaces. These micromotions enhance the osteogenic activity and formation of callus. After a callus is formed (e.g. 6 weeks postop), the clinician could then noninvasively lock the compliant mechanism through a wireless actuator, such as a magnetostrictive element that is actuated through an external device. When the compliant mechanism is locked, the stiffness of the implant at the fracture site increases which may provide the mechanical stability required for solid boney fusion.

Another set of embodiments includes smart implants that electrically-induce osteogenesis. Active configurations include smart implants with embedded electrodes on their surfaces that facilitate electrical stimulation with energy delivered to the implant through a storage element or wireless transfer mechanism. Passive configurations deliver stimulation directly through energy harvested from daily activities. Power harvesting mechanisms in passive embodiments may include piezoelectric stacks, bimetallic structures (triboelectric generators), electromagnetic harvesting (e.g. RF), and thermoelectric harvesting.

Another set of embodiments includes smart implants that actively or passively generate electromagnetic fields. Active generation may occur through the use of inductive coils or antennas embedded within the implant or attached to its surface.

Another set of embodiments includes smart implants that biochemically induce osteogenesis through controlled release of a therapeutic agent. Release mechanisms may include pump actuation, iontophoretic transport, thermal actuation (e.g. thermo-responsive hydrogel), and optical actuation, including use of a hydrogel that is responsive to stimulus from NIR or UV light. Osteogenic therapeutic agents may be released from an internal reservoir within the implant and may include of recombinant human bone morphogenic protein (e.g. rhBMP-2) as well as mesenchymal stem cells (MSCs) mixed with a carrier.

### Sensing

Sensing of bone growth activity after surgery is also of clinical significance. One method of assessing bone growth activity is through sensing the density or presence of boney tissue. These sensors may include electrical impedance spectroscopy (EIS) sensors, RF interferometry sensors, and ultrasound sensors.

Another method of sensing bone growth activity is through the electrochemical detection of bone remodeling biomarkers. Electrochemical biosensors typically include of a 3-electrode cell that is stimulated and measured using a potentiostat circuit. The working electrode is functionalized with an enzyme, antibody, antigen, or other marker-specific compound that enables electrochemical reactions to only occur when the target analyte binds with the functionalized layer. The smart implant sensor could be functionalized to detect a number of biomarkers of bone formation, bone resorption, and bone turnover regulation. Biosensors for sensing biomarkers of bone formation adjacent to a smart implant include total alkaline phosphatase (ALP) biosensors, bone-specific alkaline phosphatase (BALP) biosensors, osteocalcin (OC) biosensors, procollagen type 1 N-terminal propeptide (P1NP) biosensors, and procollagen type 1 C-terminal propeptide (P1NP) biosensors. Smart implant sensors for measuring biomarkers of bone resorption include hydroxyproline (HYP) biosensors, hydroxylysine (HYL) biosensors, deoxypyridinoline (DPD) biosensors, pyridinoline (PYD) biosensors, bone sialoprotein (BSP) biosensors, osteopontin (OP) biosensors, tartrate-resistant acid phosphatase 5b (TRAP 5b) biosensors, carboxy-terminal crosslinked telopeptide of type 1 collagen (CTX-1) biosensors, amino-terminal crosslinked telopeptide of type 1 collagen (NTX-1) biosensors, and cathepsin K (CTSK) biosensors. Smart implant sensors for measuring biomarkers of bone turnover include receptor activator of NF-kB ligand (RANKL) biosensors, osteoprotegerin (OPG) biosensors, Dickkopf-1 (DDK-1) biosensors, and sclerostin biosensors.

Another method of sensing fusion progress is through the measurement of load changes on the implant. As the bone forms, it shares loads with the implant components. Over time, the loading on the implants should decrease as bone grows as an indicator of fusion progress. Embodiments of sensors embedded within smart implants for load sensing include capacitance-based strain gauges, resistance-based strain gauges, piezoelectric force sensors, multiaxis load cells, force-sensitive resistor (FSR) sensors, FSR matrix pressure maps, and fiber Bragg grating (FBG) strain/force sensors.

### Infection

### Infection Following TKA

Periprosthetic joint infection (PJI) is one of the most feared complications in joint arthroplasty due to the ineffectiveness of antibiotics, invasive treatment options, and relatively high annual mortality rate of 4%. If caught early enough, antibiotics and natural immune responses are very effective at intercepting the free-floating bacteria within the surgical site. However, antibiotics are remarkably ineffective at eradicating bacteria within biofilm on the surface of the implant.

Biofilm develops as bacteria adhere to and colonize on the surface of an implant. The biofilm layer serves as a biochemical fortress that prevents penetration of antibiotic agents. In fact, it has been reported that 500-5000 times the concentration of antibiotics are required to have the same effectiveness on biofilm bacteria as compared to free-floating planktonic bacteria. As a result, the most common treatment for PJI is highly invasive two-stage revision. This treatment involves an initial operation to remove the septic implant and debride the surgical site and a second procedure to place new implant components. Although two-stage revision is the most common treatment option for PJI, the success rate has been reported to be a mere 85%. In addition, the risk of reinfection following revision for PJI has been reported to be 9% compared to 1-2% following the primary procedure. Also, the annual mortality rate has been reported to be as high as 14% following two-stage revision. In summary, PJI presents a rare but persistent, life-threatening complication with grossly insufficient treatment options.

Smart implants could provide surgeons and their patients the ability to monitor, detect, and diagnose PJI. Some ways to address PJI with a smart implant include monitoring infection-related parameters via integrated sensors. Electronics on-board the implant would collect and transmit physiological data such as temperature, fluid pressure, and joint stiffness. This data could be streamed to a patient engagement app on a smartphone or uploaded to the cloud to enable remote monitoring by the clinical team. A more valuable solution would incorporate an automated detection algorithm to alert the patient or physician of signs of infection. This would enable the clinician to react early and control the infection before severe symptoms develop. In addition, a sophisticated and clinically validated model could provide automated infection diagnosis directly to the physician.

Smart implants could also play a role in preventing and eradicating PJI to increase the effectiveness of antibiotics and reduce the prevalence of two-stage revision procedures. For instance, the implant could actively prevent adhesion of the bacteria to the implant surface thereby preventing biofilm development and antibiotic-resistant infection. This could be accomplished through active electrostatic repulsion facilitated by electrifying the conductive implant surface with a constant negative charge. This preventative therapy could be delivered in the post-acute period through completion of the antibiotic regimen. The previously described sensors and detection algorithm could also be incorporated to enable long-term monitoring of biofilm and infection development. If a late-stage infection is detected, the clinician could activate a moderate burst dose of electrical stimulation to detach the biofilm from the implant surface and expel the bacteria for effective interception by the antibiotic. Electrical stimulation could also facilitate an electricidal effect to destroy the infection-related bacteria without the need for antibiotics. Combining each of these solutions in a closed-loop configuration could then enable a fully autonomous smart TKA implant capable of preventing, detecting, and eradicating PJI.

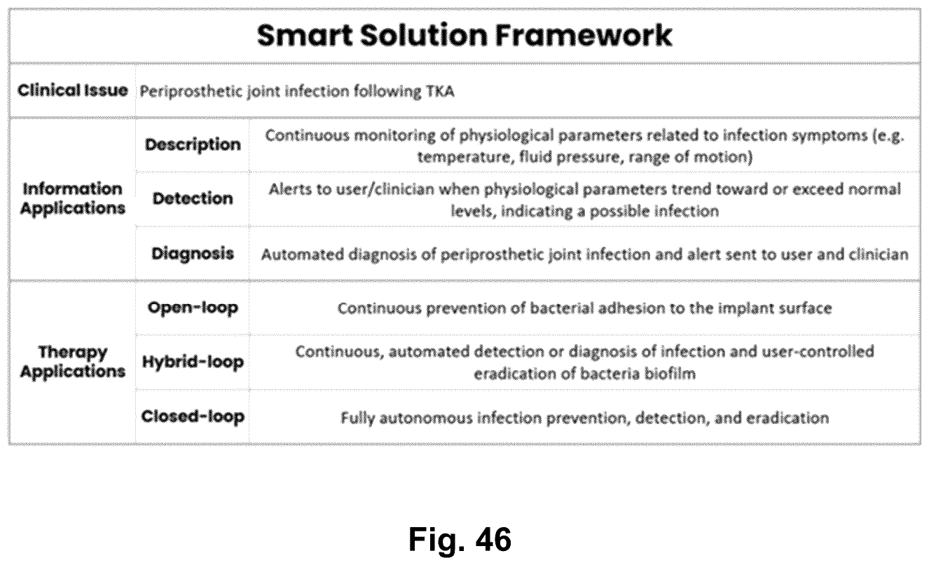

FIG. 46 illustrates a smart solution framework chart for periprosthetic joint infection following TKA, according to some embodiments of the present disclosure.

### Additional Applications and Embodiments for Infection

Beyond knee arthroplasty, infection is a major concern in nearly all musculoskeletal surgeries. In spine and trauma, these infections are referred to as deep surgical site infection (DSSI) and in joints they are referred to as periprosthetic join infection (PJI). The dynamics and effects of infection, as described above, may be similar across all device-related biofilm infections. The primary difference may be the causal microbial species. For example, in PJI of the knee S. aureus (e.g. MRSA) is one of the most commonly cultured species, however, in spine C. acnes is one of the most common pathogens. Due to the similarities in the pathology of infection and physiology of biofilms, similar approaches may be used to detect and combat implant infection across all musculoskeletal applications. This section outlines possible solutions to address these implant infections in spine, trauma, and joints.

### Detection

Early detection of infection and infection-causing biofilms is of critical importance for enabling minimally invasive treatment. Often, infections are not diagnosed until patients report symptoms to their physician, which can be well after biofilms are established. Aside from the direct effects of the infection, the presence of biofilm on the implant components can also inhibit their intended function and prevent proper bone growth. For example, one study reported that infection was diagnosed with intraoperative cultures in 10% of lumbar pseudarthrosis revision surgeries where infection was not symptomatic or suspected. This section proposes several methods and embodiments of smart musculoskeletal implants for detection of infection. In each of these embodiments, the smart musculoskeletal implant contains one or more sensors integrated within or attached to its surface to perform the detection function.

One set of embodiments for infection detection includes measurement of presence and/or amount of biofilm on the implant. Methods of measuring the physical presence of the biofilm layer include electrochemical impedance spectroscopy (EIS), RF sensing, optical sensing, and ultrasound sensing. Additional electrochemical sensing techniques could be employed to analyze the chemical dynamics of the biofilm microenvironment relative to the anatomic macroenvironment, including dissolved oxygen chronoamperometry (CA) sensing and pH open circuit potential (OCP) sensing.

Another set of embodiments involve measurement of biomarkers of infection and inflammation. Some of these embodiments integrate serum and synovial electrochemical biosensors including white blood count (WBC) biosensors, erythrocyte sedimentation rate (ESR) biosensors, C-reactive protein (CRP) biosensors. Procalcitonin (PCT) biosensors, interleukin 6 (IL-6) cytokine biosensors, interleukin 8 (IL-8) cytokine biosensors, neutrophil lymphocyte ratio (NLR) biosensors, D-dimer biosensors, albumin biosensors, globulin biosensors, alpha-defensin biosensors, and leukocyte esterase biosensors. Other embodiments integrate temperature sensing methods including measurement of temperature of the external tissue/fluid environment, bone-implant interface, and interior of the implant. Optical methods could also be utilized to measure the color and turbidity of the surrounding fluid or tissue (e.g. synovial fluid). Electrochemical techniques could be used to measure the pH of the surrounding fluid or tissue. Finally, pressure sensors could be used to measure the pressure of the surrounding fluid or tissue as a marker of inflammation.

The most direct method of detecting infection relies on detection of the pathogen on the surface of the implant. Relevant bacterial species that can be detected with smart sensors include Staphylococcus species, including Staphylococcus aureus and Coagulase-negative Staphylococci (CoNS) species, Streptococcus species, Enterococcus species, and others. Staphylococcus aureus sub-species include Methicillin-resistant Staphylococcus aureus (MRSA) and Methicillin-sensitive Staphylococcus aureus (MSSA). Coagulase-negative Staphylococci (CoNS) include Staphylococcus epidermidis, Staphylococcus lugdunensis, Staphylococcus capitis, Staphylococcus warneri, and Staphylococcus auricularis. Streptococcus species include Streptococcus agalactiae, Viridans group streptococci, Streptococcus mitis group, Streptococcus anginosus group, Streptococcus pyogenes, Streptococcus pneumoniae, and Streptococcus dysgalactiae. Enterococcus species include Enterococcus faecalis, Enterococcus faecium, and Vancomycin-resistant Enterococcus (VRE). Other relevant species include Pseudomonas aeruginosa, Escherichia coli, and Cutibacterium acnes. Methods of detecting these target bacterial analytes include optical biosensors, mechanical biosensors, and electrochemical biosensors. Optical biosensors for the detection of specific bacteria include fluorescence-based optical biosensors, thin-film optical interference spectroscopy biosensors, surface-enhanced Raman scattering (SERC) biosensors, surface plasmon resonance (SPR) biosensors, and whole-cell microscopy imaging and recognition. Mechanical methods include quartz crystal microbalance (QCM) biosensors, piezoelectric-excited millimeter-size cantilever (PEMC) biosensors, and dynamic force microscopy (DFM) biosensors. Electrochemical methods include potentiometric electrochemical biosensors, aerometric electrochemical biosensors, and impedimetric electrochemical biosensors.

This sub-section describes additional detail into the design of the electrochemical biosensors in the present disclosure. Regarding the construction and assembly configuration, the biosensor electrodes may be printed directly on the surfaces of the implant components using conductive biocompatible inks, electrodeposited on printed, molded, or machined features on the implant components, or formed on separate components that are integrated as an assembly with the implant components. These assembly configurations may include thin-film electrodes formed as a flexible circuit on a polyimide or PTFE substrate or wire-based leads over-molded or coated with a flexible silicone or polyurethane.

### Treatment

Mechanisms to prevent and eradicate infection and infection-causing biofilms may be integrated within the smart implant systems to enable non-invasive, remote, and even autonomous treatment to eliminate the need for surgical intervention. Relevant treatment mechanisms include electrochemical, mechanical, thermal, and pharmaceutical methods.

Once set of treatment embodiments rely on passive and active electrochemical mechanisms. Passive electrochemical methods include the use of a micro-cell battery array coating on the surface of the implant components comprised of a dot matrix of silver and zinc, which may be formed by printing of conductive inks, application of a thin-film layer, or embedding of rods. Active electrochemical methods include the controlled generation of antimicrobial compounds, controlled generation of gases to mechanically release the biofilm, interference of quorum sensing signals, electrostatic repulsion, and electrocution. Systems which electrolytically generate antimicrobial compounds rely on voltage-controlled electrical stimulation and can be configured to produce chlorine gas, hypochlorous acid, hydrogen gas, hydrogen peroxide, and sodium hypochlorite. When controlled effectively, these compounds can be generated in concentrations that are effective at killing bacteria on the surface of implant components while not disturbing or harming adjacent tissues. Another active electrochemical technique involves mechanically releasing the biofilm from the surface of the implant by electrolytically generating gases at the interface between the biofilm and implant. These gases can be generated similar to the previously described methods through voltage-controlled stimulation and may include chlorine gas and hydrogen gas. Another method of releasing the biofilm from the surface of the implant involves electrostatic repulsion of the negatively-charged bacterial cells with a voltage-controlled or current-controlled stimulation. By negatively charging the surface of the implant, the negatively charged bacterial cells will experience an opposing electrostatic force which will prevent adhesion to the implant micro surfaces, the first stage of biofilm formation. Another method of preventing or limiting biofilm growth involves the intentional disruption of communication signals between bacterial cells, known as quorum sensing. By generating an electrical signal with voltage-controlled or current-controlled stimulation, the communication signals between attached and planktonic bacterial cells can be disrupted and interfered preventing further accumulation of cells and formation of biofilms. Finally, a method of eliminating bacterial cells within established biofilms involves disruption of the cellular wall structure using electrocution. When properly controlled, electrical stimulation can be delivered to adhered biofilm in a magnitude that can effectively eradicate the microbial cells while not presenting harm to adjacent tissues or inducing an immune response.

Another set of treatment embodiments employ mechanical actuators embedded within the internal structure or surface of implant components. These mechanical methods include controlled release of biofilm from the implant surface using sonication (>20 kHz) generated from piezoelectric actuators, release via vibration (<1 kHz) generated from eccentric rotating mass (ERM) actuators, and removal via a brush/wiper mechanism.

Another set of treatment embodiments include the controlled release of antibiotic agents, including antibiotic drugs (e.g. gentamicin, vancomycin, tobramycin) and weak acids (e.g. acetic acid, hypochlorous acid). These agents may be stored within a reservoir within the interior of the implant or within a hydrogel coating on the surface of the implant. The agents may be channeled from an internal reservoir to the surface of the implant using integrated tubing manifold assembly or using a manifold structure that is built into the implant structure design and manufactured via 3D printing. The release of these agents may be actuated through mechanical pumps, iontophoretic transport mechanisms, thermo-responsive hydrogels activated with a thermal generator, NIR-responsive hydrogels activated with a NIR light source, and UV-responsive hydrogels activated with a UV light source (e.g. LED).

Additionally, the surface of the implant could be heated using a thermal generation device (e.g. resistive heater or magnetic heater) in a very controlled manner that enables eradication of the bacterial biofilms while not damaging the surrounding tissues.

### Aseptic Mechanical Complications

### Mechanical Loosening Following TKA

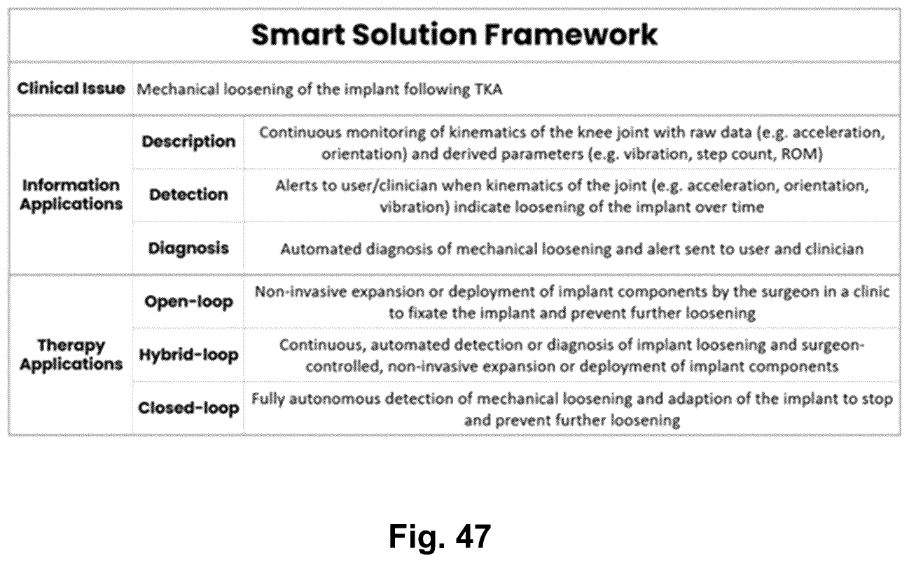

FIG. 47 illustrates a smart solution framework chart for mechanical loosening of the implant following TKA, according to some embodiments of the present disclosure.

Aseptic loosening is the mechanical loosening of a prosthetic joint without evidence of infection. Aseptic loosening may be asymptomatic in early stages but is associated with localized pain around the implant components aggravated by weightbearing and other activity-related factors. Aseptic loosening is primarily caused by a local inflammatory response that results in bone resorption at the interface between the implant and/or bone cement. However, loosening may also be attributed to poor adhesion between the implant and the cement. These effects may be the result of implant factors such as wear debris, surgical factors such as ligament balance or cement technique, and patient factors such as osteoporosis or body mass index (BMI). Aseptic loosening cannot be prevented or treated with nonsurgical measures and is the most common cause of failure and revision following TKA. The tibial component is most often affected and can be treated with an isolated tibial revision or full component revision. In either case, a revision tibial component with tibial stem or tibial stem extension is typically used to increase the implant-bone interface. Similarly, revision femoral components often include keels or femoral stems to increase fixation surface area and stability.

Smart implants may provide the ability to monitor, detect, and diagnose aseptic loosening to enable early, less-invasive treatment options. The most basic application of smart implants to address aseptic loosening following TKA would be to measure kinematics of the knee joint to monitor recovery of the patient and evaluate performance of the prosthesis. Onboard sensors could collect raw data such as acceleration and orientation of the tibial and femoral components. Additional biomechanical parameters, such as vibration, step count, and range of motion (ROM) could be derived through processing of the raw data. To provide further value, detection algorithms could be incorporated into the system to alert the clinician of trends in the kinematic data that correlate to the occurrence of aseptic loosening. In addition, a model could provide automated diagnosis directly to the care team. Although there may be no existing nonsurgical treatment options, early detection and/or diagnosis could be very valuable in providing surgeons early, less-invasive treatment options for the patient, such as a partial revision, before the pathology causes systemic harm to the entire joint.

Smart implants may also prevent, stop, and even reverse the development of aseptic loosening to reduce the prevalence of revision TKA procedures. Smart tibial and femoral components could be equipped with stimulation electrodes to create a continuous negative electrical potential at the implant-bone and/or cement-bone interface to accelerate boney on-growth and increase contacting bone density. Direct current stimulation may exploit the natural electrostatic bone modeling behaviors by signaling bone growth in areas with electronegative potential and bone resorption in areas with electropositive potential. By artificially inducing an electronegative field at the prothesis interface, it may be possible to strengthen the boney interface to prevent aseptic loosening from occurring in the first place. If loosening does develop, it may also be possible to induce sufficient osteoblast activity to counteract and even reverse the osteolytic response caused by local inflammation. The smart TKA implant could be offered in an open-loop, hybrid-loop, or closed-loop configuration. In the open-loop embodiment, the implant could deliver a continuous stimulation therapy in the immediate post-operative period. If coupled with the previously described detection capabilities, the surgeon could be alerted of loosening progression and deliver a higher dose of stimulation to stop and reverse the effects of the disease. In the most complex configuration, this treatment could be delivered autonomously without dependence on the patient or provider. In either case, smart implants may provide the first and only nonsurgical treatment option for aseptic loosening which could eliminate or delay the need for revision procedures. The smart implants may improve the quality of life for patients, reduce complication rates for surgeons, and minimize the financial burden on health systems.

### Mechanical Loosening Following THA

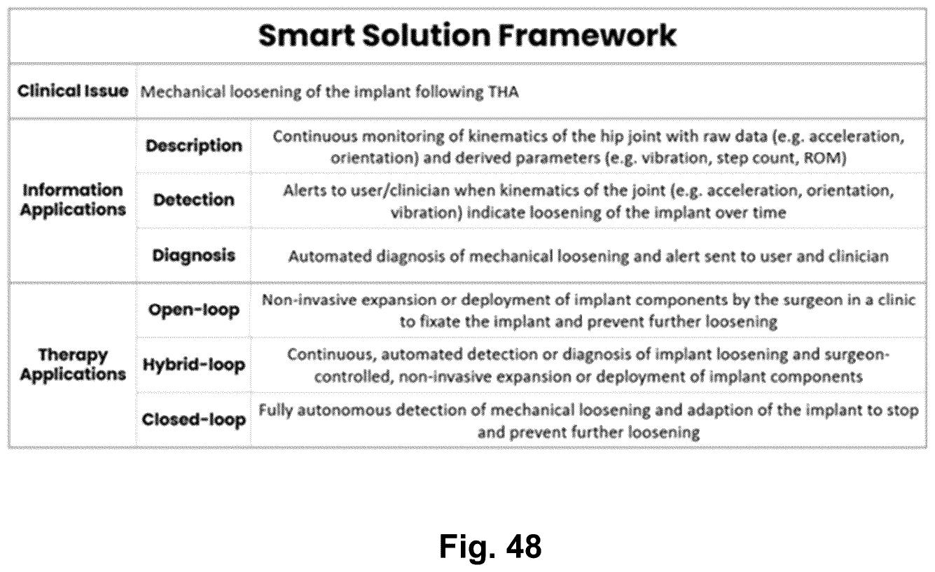

FIG. 48 illustrates a smart solution framework chart for mechanical loosening of the implant following THA, according to some embodiments of the present disclosure.

Mechanical loosening of the implant, specifically aseptic loosening, has been reported as the second most common cause of revision THA. The etiology for aseptic loosing of hip and knee prostheses is very similar and includes mechanical and biological factors. The mechanical factors include the strength of the cement, strength of the cement-implant interface, strength of the cement-bone interface, design of the implant, and loading conditions determined by the surgical execution. The primary biological factor associated with loosing of the femoral stem has historically been attributed to the normal process of endosteal enlargement of the femur during aging, particularly in osteoporotic aging women.

More recently, the biological factors in aseptic loosening have been attributed to bone resorption caused by stress shielding. According to Wolff's Law, bone changes shape and structure in reaction to changing mechanical loading conditions. Although Wolff's Law is a necessary and vital biological mechanism in musculoskeletal surgery, the strain-adaptive phenomenon can have a negative consequence for total hip replacement, which alters the loading condition of the hip joint. Prior to a primary hip replacement, load is transferred from the femoral head and epiphysis downward through the metaphyseal cortical bone. After the epiphysis is removed and the femoral stem is placed in the intramedullary canal, load is transmitted from the head of the femoral stem downward through the implant and then transferred to the adjacent metaphasis through the implant-bone interface. The implant shares a majority of the load that was previously transferred directly through the bone, which causes an effect known as stress shielding. Stress shielding causes bone stresses to be less than normal in certain regions of the femur and results in resorption in accordance with Wolff's Law. Bone resorption creates a gap between the implant-bone and cement-bone interfaces directly through loss of bone material and indirectly through weakening of bone material. This gap permits loss of rigid fixation and loosening that can cause further resorption and loosening through a positive feedback loop.

Similar to aseptic loosening following TKA, there are no nonsurgical treatment options for aseptic loosening of a hip prosthesis, however, early detection and intervention can limit effects of the complication and required invasiveness of the revision. Smart implants may provide the ability to monitor, detect, and diagnose aseptic loosening to enable early, less-invasive treatment options. a function of smart implants that may address aseptic loosening following THA includes measurement of kinematics of the hip joint to monitor recovery of the patient and evaluate performance of the prosthesis. On-board sensors could collect raw data such as acceleration and orientation of the femoral component relative to the pelvis. Additional biomechanical parameters, such as vibration, step count, and ROM could be derived through processing of the raw data. Additionally, detection algorithms could be incorporated into the system to alert the clinician of trends in the kinematic data that correlate to the occurrence of aseptic loosening, and a validated model could provide automated diagnosis directly to the care team. Through early detection, surgeons could evaluate the progression of loosening and intervene before catastrophic damage to the bone occurs.

In addition to early detection, smart implants may also provide a therapeutic function to stop and prevent further development of aseptic loosening to reduce the number of revision THA procedures. It may be unlikely that the stress shielding effect of the femoral stem can be avoided without a fundamental shift in implant design (e.g. addition of a flange feature to transfer load to the cortical ring) and surgical ideology, however, conventional implant designs may be augmented with smart technologies to limit aseptic loosening caused by stress shielding. For example, the proximal portion of the femoral stem could contain an expansion mechanism to adapt the fixation of the stem to a developing gap at the implant-bone interface. Various mechanism configurations could be employed, including a continuous surface expanding mandrel mechanism or a mechanism with distinct expanding surfaces similar to a 3-point bore micrometer. The expansion mechanism could be actuated wirelessly via shape memory alloys to enable a nonsurgical treatment option. An open-loop configuration of the smart THA implant could be adjusted in a clinic setting with accompanying radiographs. If coupled with the sensing and loosening detection capabilities, the implant could provide real-time feedback to the clinician regarding the impact of the adjustment on the mechanical stability of the implant components. This detection, adjustment, and verification process could also be controlled autonomously in a closed-loop configuration. Regardless of the embodiment, such solutions would provide the first and only nonsurgical treatment option for mechanical loosening following hip replacement and likely reduce the incidence and burden of revision surgery.

### Dislocation Following THA

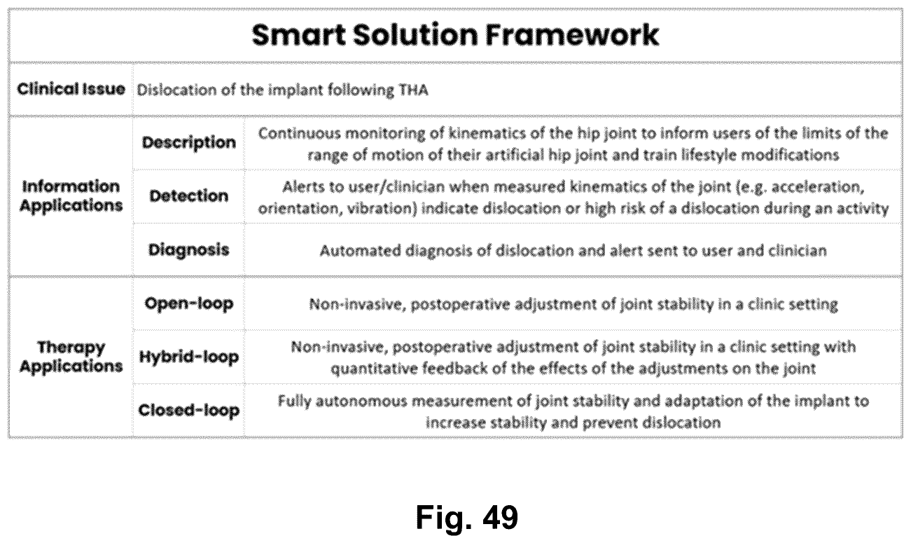

FIG. 49 illustrates a smart solution framework chart for dislocation of the implant following THA, according to some embodiments of the present disclosure.

Dislocation is one of the most common complications following THA. Dislocation occurs when the femoral head dislodges from the liner of the acetabular cup. Several surgical factors, including surgical approach, soft-tissue tension, component positioning, impingement, head size, liner profile, and surgeon experience, can contribute to dislocation. The cause is typically complex and multifactorial rather than attributed to an acute fall or traumatic event. Dislocations within weeks or months of the operation correlate to problems with soft tissue tension, component malposition, infection, or patient noncompliance. Dislocations beyond a year often suggest stretching of the soft tissues or wear of the liner component. Patients are usually immediately aware when a dislocation occurs due to intense local pain and loss of motion in the joint. Dislocation can often be treated non-invasively through closed reduction and bracing, but recurrent dislocation typically requires revision surgery to address the underlying issue. Revision surgery may include resection of osteophytes or modification of implant geometry to correct impingement, increase of the implant offset or tightening of the soft tissue tension to correct joint instability, and modification of the cup location/orientation to correct malposition.

Although the occurrence of dislocation is usually immediately obvious, the behaviors and activities that increase risk of dislocation are often not apparent to the patient. Smart implants may provide value to the patient and provider by preventing dislocation through awareness and lifestyle modification. For example, a smart THA implant may continuously monitor the kinematics of the hip joint to inform users of the range of motion (ROM) limits of their artificial joint. This information could be coupled with a physical or occupational therapy protocol to train patients how to perform activities of daily living in a safe manner. With the addition of a detection algorithm, a patient could be warned when the measured kinematics indicate high risk of dislocation during activity. In a diagnostic configuration, the implant could automatically alert the care team when a dislocation occurs, which could be especially valuable and even lifesaving for elderly patients who live independently. The description and detection applications outlined above add value in limiting patient-related factors and do not address surgical factors. Because surgical factors must be addressed intraoperatively, it may be more applicable to address these issues with other solutions such as enabling technologies and procedural solutions.

Smart implants may also play a therapeutic role in preventing dislocation by correcting soft tissue instability. For example, artificial ligaments with adjustable length and stiffness could be used in combination with the femoral and acetabular components to correct joint instability in a non-invasive manner. In one embodiment, the smart artificial ligament would include of two tethers joined at the center with an actuator and anchors on each of the distal ends. The actuator could be length-adjustable to tension the artificial ligament assembly in a constant stiffness configuration or stiffness-adjustable to tension the assembly in a constant length configuration. Non-invasive actuation could be accomplished wirelessly via shape memory alloys to allow for adjustment in a clinical setting. A full construct might include of three smart artificial ligaments arranged to compliment the iliofemoral, pubofemoral, and ischiofemoral ligaments, with one end of each artificial ligament stapled to the greater trochanter and the other end affixed to the pelvis. The three artificial ligaments could be wireless adjusted to accomplish the desired joint stability and range of motion. These devices could also be used to intentionally limit joint motion during the post-acute period as an internal alternative to cumbersome and uncomfortable hip braces. The smart THA implant with artificial ligaments could be offered in open-loop, hybrid-loop, or closed-loop configurations with each offering increased levels of feedback and autonomous functionality.

### Adjacent Segment Disease

### Adjacent Segment Disease Following Lumbar Interbody Fusion

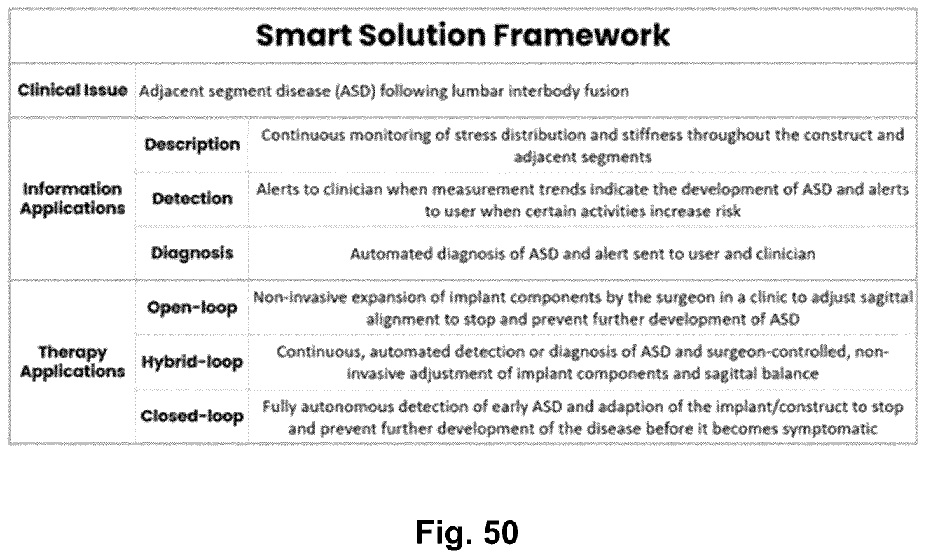

FIG. 50 illustrates a smart solution framework for adjacent segment disease (ASD) following lumbar interbody fusion, according to some embodiments of the present disclosure.

ASD has been reported as the second most common indication of revision lumbar fusion. Adjacent segment disease is a broad and complex pathology associated with the instability and degradation of adjacent levels following a spinal fusion procedure. Like most musculoskeletal pathologies, the etiology of ASD is multifactorial and has not been attributed to a single cause. Changes in intradiscal pressure, soft tissue disruption, facet joint violation, and sagittal malalignment have been proposed as major factors. Each factor shares a common effect of increased stresses on the adjacent levels which leads to progressive degradation of the adjacent disc and subsequent stenosis. Unfortunately, there are not any existing nonsurgical treatment options for ASD. Patients presenting with radiographic ASD are typically monitored until severe symptoms develop, at which point the construct is revised and typically extended to fuse the adjacent level. Though not every lumbar fusion patient develops ASD, patients with the disease often will have recurrent pathology and require multiple revisions throughout their lifetime as adjacent levels degrade following each procedure.

Smart implants could offer insight into when, how, and why ASD develops through in situ data collection. The stress distribution and stiffness of the construct could be continuously monitored through load cells integrated into the interbody device, rods, and screws. Though this data would only measure loading of the fused construct, correlations could be drawn between the surgical technique, placement of the selected implants, and diagnosis of the disease. To directly measure the effects of fusion on the adjacent anatomy, strain sensors could be placed minimally invasively in the adjacent disc spaces to measure intradiscal pressures and mobility of the adjacent segment could be measured through inertial measurement units (IMU) on the adjacent vertebral bodies. These sensors could enable real-time monitoring, detection, and diagnosis of ASD and contributing factors. Rather than providing significant value to each individual patient at the onset, this information would likely benefit future populations of patients by enhancing clinical understanding of the disease, the contributing factors, and strategies to minimize incidence.

Malalignment, particularly in the sagittal plane, is a major contributing factor to ASD that continues to be addressed with new technology and surgical techniques. Global alignment and sagittal balance are increasing focuses of conversation in the orthopedic community due to the recognition that treatment of a singular anatomical region can impact the biomechanics of the entire skeleton and a patient's future wellbeing. Some interbody implants may provide a variety of lordotic correction options, including our own innovative designs with height expansion and adjustable lordotic angle. In addition, preoperative planning tools, provide surgeons the ability to measure the correction required to achieve their desired outcome and select the properly sized implants and techniques. Robotic navigation systems, such as ExcelsiusGPS, assist surgeons in accurate anterior positioning of these lordotic implants to maximize segmental lordosis. Procedural techniques, such as prone lateral, may be explored to maximize lordotic correction using the aid of gravity and the patient's soft tissue. Despite these advances, there is some level of unpredictable settling of the implants within the anatomy that occurs following surgery resulting in loss of lordosis and a negative impact on global alignment. In fact, some studies have reported postoperative segmental lordosis equivalent to half of the designed implant lordotic angle. For this reason, surgeons may overcorrect the patient on the table with the expectation that their anatomy will settle to the desired alignment, but sustained overcorrection, particularly in adult deformity correction, can place the patient at risk of other complications such as proximal junctional kyphosis (PJK).

There is an opportunity for smart implants to further prevent or decelerate the development of ASD via postoperative, noninvasive adjustment of lordotic correction. This function may be accomplished through a wireless actuator integrated in the expansion mechanism of an expandable interbody implant. The wireless actuator could be a ratcheting mechanism made of a shape memory alloy that rotates a screw-based expansion mechanism or a linear ratchet that directly expands the implant. Regardless of the embodiment, the implant could be placed and manually expanded intraoperatively like normal and postoperatively adjusted after the implants and anatomy settle. Moderate adjustment may be achieved following single stage procedures with counteracting posterior fixation in place, and maximum impact would likely be realized in two-stage procedures where implants could be noninvasively adjusted before posterior fixation is locked in place.

Smart implants could aid in load sharing across adjacent levels to limit the stresses directly transferred through the adjacent facet joints and discs, particularly in adult deformity correction. Ligament augmentation is a relatively new technique employed to reduce rates of PJK and ASD. In this technique, the posterior ligaments are augmented by placing and tensioning a polyester fiber tether (i.e. Mersilene suture) between a hole created in the superior adjacent spinous process and a crosslink connector attached to the posterior fixation construct. Initial studies attribute the associated reduced rates of PJK and ASD to stress sharing and stability that the artificial ligaments provide in counteracting the disrupted posterior soft tissues. Therefore, this technique has the most promise in open or mini-open adult deformity procedures and less applicability for minimally invasive procedures. A limitation of this technique is that the tension of the artificial ligament is fixed and cannot be adjusted postoperatively and the tensioning of the tether is very subjective, based on feel. There is an opportunity to create a smart artificial interspinous ligament with noninvasive, postoperative adjustability and load sensing. The design of the smart ligament could be very similar to the embodiments previously discussed for THA, with a shape memory alloy actuator for wireless adjustment.

Together, the discussed smart interbody spacer and smart artificial ligament devices could offer unparalleled postoperative, noninvasive adjustability and assessment of global alignment and load distribution. Each device could be configured in open-loop, hybrid-loop, or closed-loop configurations. Regardless of the configuration, these smart implants could provide the information and control for surgeons achieve their postoperative correction goals, limit the development of ASD, and decrease the occurrence of revision procedures.

### Pain Management

Persistent postoperative pain is a major driver of insufficient patient-reported outcomes after surgery. For example, lower back pain after lumbar fusion and knee pain after total knee arthroplasty are often reported by patients may not be easily explained or addressed by clinicians. One possible cause is damage to minor neural structures embedded within the bone that is modified as part of the surgery, however, the severity and incidence of this pain is inconsistent from patient to patient. There is an opportunity for smart musculoskeletal implants to play a role in post-operative pain management by quantifying the pain level and enabling non-invasive, targeted pain treatment.

One method of quantifying pain is by integrating biosensors for pain and stress biomarkers into the smart implant components. Biosensors for biomarkers of pain include quinolinic acid biosensors, kynurenic acid biosensors, mercapturic acid biosensors, 5-hydroxyindoleacetate biosensors, and vanilmandelate biosensors. Biosensors for biomarkers of stress include cortisol biosensors. The biosensor mechanism and embodiments could be similar to what was previously described in this disclosure with the primary difference being that the working electrode or sensitive aspect of the sensor is functionalized to specifically detect the presence and concentration of the aforementioned biomarkers.

One method of treating pain with a smart musculoskeletal implant is through electrical stimulation of the adjacent neural elements. This may include or be similar to spinal cord stimulators and nerve stimulator devices. Unlike spinal cord stimulators and nerve stimulator device, some embodiments include a stimulation system integrated within the structure or assembly of a musculoskeletal implant, such as a tibial tray or lumbar interbody spacer. Stimulation electrodes could be arranged along the surface of the implant to facilitate generation of the electrical signal that stimulates the adjacent nerves. Additionally, this electrode array could also be shared with other implant functions, such as bone growth stimulation, infection treatment, and electrochemical sensing. The system could be capable of generating a range of stimulation treatments depending on the surgeon and patient preference. Relevant protocols include low stimulation frequencies <200 Hz that produce a paresthesia affect that numbs or masks pain as well as high stimulation frequencies ˜10,000 Hz that treat the pain without paresthesia effects.

Another approach to treating pain with a smart musculoskeletal implant is through controlled, targeted delivery of pharmaceutical pain drugs to the adjacent tissues. Targeted delivery enables higher concentrations of drugs, such as opioids, to be used than normally safely possible using oral methods. These drugs could be stored within a reservoir within the interior of the implant or within a hydrogel coating on the surface of the implant. The agents could be channeled from an internal reservoir to the surface of the implant using integrated tubing manifold assembly or using a manifold structure that is built into the implant structure design and manufactured via 3D printing. The release of these agents could be actuated through mechanical pumps, iontophoretic transport mechanisms, thermo-responsive hydrogels activated with a thermal generator, NIR-responsive hydrogels activated with a NIR light source, and UV-responsive hydrogels activated with a UV light source (e.g. LED).

### Oncology

In some cases where tumors are resected from bone or tissue adjacent to bone, musculoskeletal implants (e.g. corpectomy cages, plates, fixation screws) may be used to stabilize the compromised skeletal structures. Smart musculoskeletal implants may play a role in post-operative surveillance and treatment of tumors by detecting the presence of cancerous tissues and cells and enabling non-invasive, targeted cancer treatment. Relevant sensing methods include sensing the impedance of the adjacent tissue and comparing the measured values to known tumorous tissue values, direct detection of cancerous cell types using electrochemical biosensors, direct detection via whole-cell microscopy imaging and recognition, and indirect detection using biosensors for cancer biomarkers.

One approach to treating cancer with a smart musculoskeletal implant is through controlled, targeted delivery of pharmaceutical chemotherapy drugs to the adjacent tumorous tissues. These drugs could be stored within a reservoir within the interior of the implant or within a hydrogel coating on the surface of the implant. The agents could be channeled from an internal reservoir to the surface of the implant using integrated tubing manifold assembly or using a manifold structure that is built into the implant structure design and manufactured via 3D printing. The release of these agents could be actuated through mechanical pumps, iontophoretic transport mechanisms, thermo-responsive hydrogels activated with a thermal generator, NIR-responsive hydrogels activated with a NIR light source, and UV-responsive hydrogels activated with a UV light source (e.g. LED).

Another approach to treating cancer with smart implants includes targeted ablation. In some embodiments, the smart implant includes an array of stimulation electrodes on or within its surface, as previously described in other embodiments. These electrodes could be selectively activated to steer and direct a high dose of stimulation to ablate the tumorous tissue.

Smart Implants that Enable Novel & Less-Invasive Procedures

### Femoral Necrosis

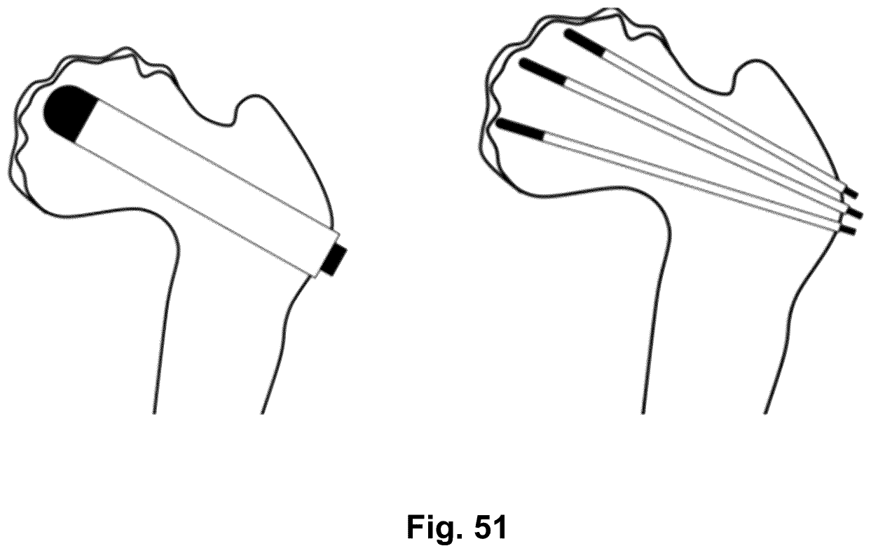

FIG. 51 illustrates rods or screws inserted within a femoral neck of a patient, according to some embodiments of the present disclosure.

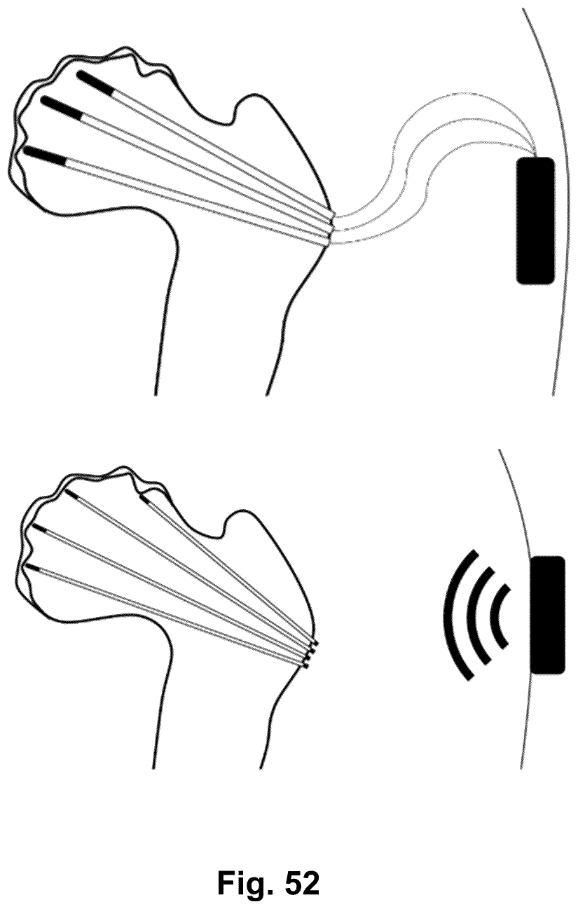

FIG. 52 illustrates rods or screws inserted within the femoral neck and is connected to a smart implant or is in contact with an external device, according to some embodiments of the present disclosure.

Femoral necrosis is a major clinical issue, particularly in smokers who have poor vascularization, that causes osteoarthritis of the hip joint and collapse of the femoral head. Treatment options for femoral necrosis are limited to bone grafting and hip arthroplasty. Smart musculoskeletal implants may be capable of directing bone growth stimulation treatment to necrotic areas, such as in the femoral head.

In some embodiments, stimulation electrodes are embedded within a porous tantalum or titanium rod or screw inserted within the femoral neck following a core decompression. The leads could be positioned such that the stimulation encourages bone remodeling at the necrotic superior surface of the femoral head while the porous rod promotes blood flow and provide structural support. Power could be provided to the device via stored, wirelessly transferred, or harvested methods previously described in this disclosure.

The rod/screw may include a structure containing one or more electrodes located at a tip of the rod/screw that is configured to deliver targeted electrical bone growth stimulation to the site of a femoral necrosis on the femoral head. FIG. 51, on the left, shows a single trochanteric screw that provides structural support to the femoral head and neck while delivering electrical stimulation functions. FIG. 51, on the right, shows smaller electrode needles/rods that function to position the stimulation electrodes near the necrotic tissue. Both embodiments may be powered through wires via an implantable module (as shown at the top of FIG. 52) or wirelessly via a wearable device (as shown at the bottom of FIG. 52).

In some embodiments, the smart implants may enable monitoring of microdiscectomy outcomes with a smart intradiscal pressure sensor patch and/or enable post-operative optimization of a smart artificial nucleus device, such as an elastomeric sac filled with a fluid which has a pressure that can be wirelessly controlled.

### Fiber Bragg Grating (FBG) Sensing

FBG sensors are a class of fiber-optic based sensors that leverage the fiber Bragg gratings principle to enable sensing of temperature, shape, strain, force, and pressure across the length of an optical fiber or array of fibers. FBG sensors are created by inscribing encoded gratings along the length of an optical fiber. When an incident spectrum of light is passed through the fiber, a specific portion of the spectrum, called the Bragg wavelength, is reflected back to the source. Inducing a strain in the fiber causes a proportional shift in the reflected Bragg wavelength. This proportionality can be calibrated to dynamically measure a variety of parameters, including shape and force. A key advantage of FBG sensors is that multiple measurement points can be fabricated as an array of independent sensors along a single fiber, enabling multiplexed and distributed measurements such as 3D shape.

FBG sensing is a compelling technology for force and shape sensing in smart implants, smart instruments, robotics, and wearable devices. Regarding smart implants, FBG fibers could be embedded around the exterior of fixation rods to enable continuous sensing of the load and strain along the length of the rod. Clinically, such sensors may enable measurement of strain in the rod during bending, the force between the locking cap, rod, and screw tulip during placement, and loading conditions along the construct during postoperative recovery. Similarly, the fibers could be embedded along the surface of bone-contacting implants, such as pedicle screws, interbody endplates, and femoral stems, to measure the total force and force distribution along the bone-implant interface. These sensing fibers could also be placed along the shafts of instruments to measure deflection during use. This application could have immense valuable during robotic pedicle preparation as an added safety measure and input to the system for active skiving compensation. This technology could also greatly enhance free-hand navigation of delicate instruments, such as MIS discectomy instruments, which are prone to tip deflection. Finally, in the context of wearables, FBG sensors could be woven into clothing, braces, or adhesive tape to enable pre-op and post-op data collection of patient mobility parameters, such as range of motion.

### Compliant Mechanisms

Compliant mechanisms are a class of mechanisms that rely on flexibility (compliance) to accomplish their function. Traditionally, mechanical designers have relied on very stiff, rigid parts connected with hinges or sliding joints to form complex mechanisms. However, things that move in nature are often very flexible and the propelled by the bending of flexible parts. Bee wings, elephant trunks, eels, spines, and flower petals are all examples of compliant mechanisms in the natural world. In addition to enabling biomimicry, compliant mechanisms also offer increased performance (e.g. high precision, low weight, low friction), lower cost (e.g. lower part count), and ability to miniaturize to the micro and nano scale.

With respect to musculoskeletal surgery, compliant mechanisms are especially compelling for the design of smart implants capable of reverse dynamization. Reverse dynamization is a mechanical manipulation regimen designed to accelerate bone healing by first allowing micromotions to encourage cartilaginous callus formation and later stabilizing the construct to prevent the disruption of neovascularization. To enable reverse dynamization, a smart implant construct may be capable of allowing micromotions for a controlled period of time and dynamic adjustment to a rigid configuration either automatically or at the request of a clinician. Compliant mechanisms may be beneficial for some of these embodiments because of the ability to design for specific stiffness, force, or motion requirements. In addition, compliant mechanisms can be designed in a bi-stable configuration to allow more than one equilibrium state with varied stiffnesses. A tunable stiffness compliant mechanism could be designed with a wirelessly actuated bi-stable switch to allow a clinician to change the state of an implant from a state permitting flexibility and micromotions to a state of rigid fixation. A mechanism such as this would particularly valuable if integrated in orthopedic trauma devices such as intramedullary nails and fracture plates.

An example of a smart implant capable of reverse dynamization may include a trauma fixation plate with material removed along its length to form a compliant mechanism section. The plate may be positioned on the bone such that the compliant mechanism section is near the fracture site and the plate would be fixed above and below the fracture site. The compliant mechanism section may enable relative motion between the portion of bone above and below the fracture line, which enables the biomechanical process of dynamization that accelerates osteogenic activity. After a period of time, the dynamization may be stopped by locking the compliant mechanism. This can be accomplished with a switch that is actuated noninvasively with a magnet.

What is claimed is:

1. A method performed by a musculoskeletal implant device, comprising:
obtaining sensor information indicating measurements taken by a sensor connected to the musculoskeletal implant device during or after implantation of the musculoskeletal implant device into a patient; and
providing feedback for controlling modification of a feature associated with the musculoskeletal implant device based on the sensor information.

2. The method of claim 1, wherein the providing of feedback for controlling modification of the feature associated with the musculoskeletal implant device comprises providing feedback for controlling modification of at least one of:
position of the musculoskeletal implant device implanted in the patient,
orientation of the musculoskeletal implant device implanted in the patient, and
expansion or contraction of a shape of the musculoskeletal implant device.

3. The method of claim 2, while the musculoskeletal implant device is being positioned, oriented, expanded or contracted in the patient, providing through a display device feedback indicating distribution of pressure loading across the musculoskeletal implant device based on the sensor information indicating load values corresponding to a plurality of locations on the musculoskeletal implant device.

4. The method of claim 1, wherein the providing of feedback for controlling modification of the feature associated with the musculoskeletal implant device comprises providing feedback for controlling modification of at least one of:
electrical stimulation of a sensor electrically connected to the musculoskeletal implant device, and
electrical stimulation of an electrode electrically connected to the musculoskeletal implant device.

5. The method of claim 1, wherein the sensor information indicates at least one of:
fusion or bone formation of a bone contacting the musculoskeletal implant device;
load values corresponding to a plurality of locations on the musculoskeletal implant device;
impedance measured from the musculoskeletal implant device in contract with tissue of the patient;
phase in voltage measured from the musculoskeletal implant device in contract with tissue of the patient;
torque on the musculoskeletal implant device;
strain on the musculoskeletal implant device;
biomarkers sensed by the musculoskeletal implant device;
distribution of a substance in or on the musculoskeletal implant device;
amount of filling of a substance in the musculoskeletal implant device; and
periprosthetic joint infection.

6. The method of claim 1, wherein the sensor information indicates periprosthetic joint infection or bone growth, and further comprising providing the feedback to control activating, increasing, and/or decreasing vibrations generated by a vibration actuator within or connected to the musculoskeletal implant device.

7. The method of claim 1, wherein:
the sensor information indicates amount of filling of a substance in or on the musculoskeletal implant device at defined locations on the implant using impedance values measured between a plurality of spaced apart electrodes at the defined locations on the implant, and
providing feedback for controlling further distribution of filling of the substance in or on the musculoskeletal implant device.

8. The method of claim 1, wherein the providing of the feedback comprises:
transmitting the sensor information to a controlling device that is external to the patient; and
providing instructions from the controlling device to a tool configured to expand shape of the musculoskeletal implant device.

9. The method of claim 1, wherein the providing of the feedback comprises:
transmitting the sensor information to a controlling device that is external to the patient; and
providing instructions from the controlling device to a circuit configured to generate electrical stimulation through the musculoskeletal implant device.

10. A musculoskeletal implant device, comprising:
at least one sensor;
processing circuitry; and
a memory containing instructions executable by the processing circuitry, whereby the musculoskeletal implant device is operable to:
obtain sensor information indicating measurements taken by the sensor connected to the musculoskeletal implant device during or after implantation of the musculoskeletal implant device into a patient; and
provide feedback for controlling modification of a feature associated with the musculoskeletal implant device based on the sensor information.

11. The musculoskeletal implant device of claim 10, wherein the providing of feedback for controlling modification of the feature associated with the musculoskeletal implant device comprises providing feedback for controlling modification of at least one of:
position of the musculoskeletal implant device implanted in the patient,
orientation of the musculoskeletal implant device implanted in the patient, and
expansion or contraction of a shape of the musculoskeletal implant device.

12. The musculoskeletal implant device of claim 11, while the musculoskeletal implant device is being positioned, oriented, expanded or contracted in the patient, provide through a display device feedback indicating distribution of pressure loading across the musculoskeletal implant device based on the sensor information indicating load values corresponding to a plurality of locations on the musculoskeletal implant device.

13. The musculoskeletal implant device of claim 10, wherein the providing of feedback for controlling modification of the feature associated with the musculoskeletal implant device comprises providing feedback for controlling modification of at least one of:
electrical stimulation of a sensor electrically connected to the musculoskeletal implant device, and
electrical stimulation of an electrode electrically connected to the musculoskeletal implant device.

14. The musculoskeletal implant device of claim 10, wherein the sensor information indicates at least one of:
fusion or bone formation of a bone contacting the musculoskeletal implant device;
load values corresponding to a plurality of locations on the musculoskeletal implant device;
impedance measured from the musculoskeletal implant device in contract with tissue of the patient;
phase in voltage measured from the musculoskeletal implant device in contract with tissue of the patient;
torque on the musculoskeletal implant device;
strain on the musculoskeletal implant device;
biomarkers sensed by the musculoskeletal implant device;
distribution of a substance in or on the musculoskeletal implant device;
amount of filling of a substance in the musculoskeletal implant device; and
periprosthetic joint infection.

15. The musculoskeletal implant device of claim 10, wherein the sensor information indicates periprosthetic joint infection or bone growth, and further operable to provide the feedback to control activating, increasing, and/or decreasing vibrations generated by a vibration actuator within or connected to the musculoskeletal implant device.

16. The musculoskeletal implant device of claim 10, wherein:
the sensor information indicates amount of filling of a substance in or on the musculoskeletal implant device at defined locations on the implant using impedance values measured between a plurality of spaced apart electrodes at the defined locations on the implant, and
provide feedback for controlling further distribution of filling of the substance in or on the musculoskeletal implant device.

17. The musculoskeletal implant device of claim 10, wherein the providing of the feedback comprises:
transmit the sensor information to a controlling device that is external to the patient; and
provide instructions from the controlling device to a tool configured to expand shape of the musculoskeletal implant device.

18. The musculoskeletal implant device of claim 10, wherein the providing of the feedback comprises:
transmit the sensor information to a controlling device that is external to the patient; and
provide instructions from the controlling device to a circuit configured to generate electrical stimulation through the musculoskeletal implant device.

19. A method performed by a medical system for controlling modification of a musculoskeletal implant device, comprising:
receiving, from the musculoskeletal implant device, sensor information indicating measurements taken by a sensor connected to the musculoskeletal implant device during or after implantation of the musculoskeletal implant device into a patient; and
transmitting modification instructions for controlling modification of a feature associated with the musculoskeletal implant device based on the sensor information.

20. The method of claim 19, wherein the sensor information indicates at least one of:
fusion or bone formation of a bone contacting the musculoskeletal implant device;
load values corresponding to a plurality of locations on the musculoskeletal implant device;
impedance measured from the musculoskeletal implant device in contract with tissue of the patient;
phase in voltage measured from the musculoskeletal implant device in contract with tissue of the patient;
torque on the musculoskeletal implant device;
strain on the musculoskeletal implant device;
biomarkers sensed by the musculoskeletal implant device;
distribution of a substance in or on the musculoskeletal implant device;
amount of filling of a substance in the musculoskeletal implant device; and
periprosthetic joint infection.
